(19) United States
(12) Patent Application Publication
(10) Pub. No.: US 2024/0181118 A1
(43) Pub. Date: Jun. 6, 2024

(54) LIGHT EMITTING ELEMENT WITH INTEGRATED IONIZER

(71) Applicant: SIGNIFY HOLDING B.V., EINDHOVEN (NL)

(72) Inventors: PAUL EMMANUEL MARTHE JULES REUSENS, EDEGEM (BE); GEORGES MARIE CALON, EINDHOVEN (NL); ARTHUR MOLLET, EINDHOVEN (NL); ROGIER GODEFRIDUS JOHANNES MARIA WASSENBERG, WAALRE (NL); THEO GERRIT ZIJLMAN, TILBURG (NL); ZHI HONG XIE, SHANGHAI (CN)

(21) Appl. No.: 18/284,922

(22) PCT Filed: Mar. 29, 2022
(86) PCT No.: PCT/EP2022/058186

§ 371 Date: Sep. 29, 2023

(30) Foreign Application Priority Data
Apr. 1, 2021 (WO) PCT/CN2021/085012
May 27, 2021 (EP) 21176272.9

(51) Int. Cl. A61L 9/22; F21S 8/04; F21V 23/00; F21V 33/00; F21Y 115/10

(52) U.S. Cl. CPC A61L 9/22; F21S 8/046; F21V 23/003; F21V 33/0004; F21Y 2115/10

(57) ABSTRACT

The invention provides a device (1200) comprising (i) a light generating device (100), (ii) an air ionizer device (500), and (iii) an airflow device (600), wherein: (A) the device (1200) comprises one or more first openings (710) and a front part (750) comprising one or more second openings (720); (B) the air ionizer device (500) is configured to generate in an operational mode positively charged particles at a first electrode (510) and negatively charged particles at a second electrode (520); (C) the airflow device (600) is configured to generate in the operational mode a first airflow (610) entraining the positively charged particles and a second airflow (620) entraining the negatively charged particles; wherein the one or more first openings (710) are configured upstream of the airflow device (600) and the electrodes (510,520); wherein a first part (721) of the one or more second openings (720) is configured downstream of the airflow device (600) and the first electrode (510), and a second part (722) of the one or more second openings (520) is configured downstream of the airflow device (600) and the second electrode (520); (D) the device (1200) comprises a light emitting area (150), from which during operation of the light generating device (100) device light (1201) escapes from the device (1200), wherein the front part (750) comprises the light emitting area (150).

### FIELD OF THE INVENTION

The invention relates to a device, which may (also) have a lighting function. The invention further relates to a light generating system comprising such device. Yet further, the invention relates to a method wherein the device may be applied.

### BACKGROUND OF THE INVENTION

Ion generators are known in the art. US2012/0028561, for instance, describes an ion generator comprising: one or a plurality of pairs of ion generating parts generating positive and negative ions; and an air guiding member in which openings releasing to the outside the positive and the negative ions generated by each pair of the ion generating parts are formed and which guides air to the opening, wherein the openings are formed in different sites in the air guiding member, and wherein the air guiding member is so constructed that the directions of ion release in the different sites are different from each other.

CN 106 051 496 A discloses an LED lamp with high-efficiency air purification and heat dissipation functions and belongs to the field of LED lighting lamps. The LED lamp comprises an LED part and a purifier part. The output end of a rectifier bridge set is connected with the LED part and the purifier part. A fan is arranged inside a shell. A vent drum is located below the fan. Air guiding fins evenly distributed are arranged on the inner side face of the vent drum. An anion generating slot is arranged inside the vent drum. An anion generating mechanism is arranged in the anion generating slot. A lower opening of a guide plate is right opposite to a heat dissipation device. The heat conducting surface of an LED lamp bead set is tightly attached to the lower end of a heat conducting plate. An annular convex plate is fixed to the upper end of the heat conducting plate. An axial through hole is formed in the middle of the heat dissipation device. The annular convex plate at the upper end of the heat conducting plate is matched with the axial through hole in the middle of the heat dissipation device. By means of the LED lamp, high-efficiency diffusion of anion air is achieved, the air purification effect is improved, meanwhile, the airflow swirling at a high speed is beneficial for high-efficiency heat dissipation of the heat dissipation device, and the service life of the LED lamp bead set is prolonged.

### SUMMARY OF THE INVENTION

In order to prevent the spread of bacteria and viruses such as influenza or novel (corona) viruses like COVID-19, SARS and MERS, it appears desirable to produce systems that provide (alternative) ways for air treatment, such as disinfection. Existing systems for disinfection may not easily be implemented in existing infrastructure, such as in existing buildings like offices, hospitality areas, etc. and/or may not easily be able to serve larger spaces. This may again increase the risk of contamination. Further, incorporation in HVAC systems may not lead to desirable effects and appears to be relatively complex. Further, existing systems may not be efficient, or may be relatively bulky, and may also not easily be incorporated in functional devices, such as e.g. luminaires.

Amongst others, the present invention proposes to combine a system having a lighting function with a system having an air treatment function, such as disinfection. A method for disinfection may be the use of air ionizers. Microorganisms may be killed by positively charged particles and/or negatively charged particles in air.

Several solutions may be possible. However, it appears that not all solutions lead to useful systems. For instance, disadvantages of monopolar and bipolar ion emitting lighting devices may be that a large space needed on the front of the lamp for ion outlet, limiting the space for lighting function. Other disadvantages appear to be solutions wherein obstruction of the light path by ionization components (needles, brushes) in the light path occur. Further, some solutions may lead to a recombination of positive and negative ions because of colliding airflows with ions of opposite polarity. Yet further, solutions may also have as disadvantage that ions cloud may only reach the close vicinity of the lamp.

Hence, it is an aspect of the invention to provide an alternative system or device for air treatment, which preferably further at least partly obviates one or more of above-described draw backs. The present invention may have as object to overcome or ameliorate at least one of the disadvantages of the prior art, or to provide a useful alternative.

In a first aspect the invention provides a device (“device” or “multi-functional device”) comprising (i) a light generating device, (ii) an air ionizer device, and (iii) an airflow device. Especially, in embodiments the device may comprise one or more first openings (“inlets”) and a front part comprising one or more second openings (“outlets” or “airflow outlets”). Further, the air ionizer device may especially be configured to generate in an operational mode positively charged particles at a first electrode and negatively charged particles at a second electrode. Yet further, in embodiments the airflow device may be configured to generate in the operational mode a first airflow entraining the positively charged particles and/or a second airflow entraining the negatively charged particles. In embodiments, the one or more first openings may be configured upstream of the airflow device and the electrodes. Especially, in embodiments a first part of the one or more second openings may be configured downstream of the airflow device and the first electrode. Yet further, in embodiments a second part of the one or more second openings may be configured downstream of the airflow device and the second electrode. Especially, in embodiments the first part and the second part may be spatially separated. Further, the device may comprise a light emitting area, from which during operation of the light generating device, device light may escape from the device. Especially, the front part comprises the light emitting area. Hence, in embodiments the device comprises (i) a light generating device, (ii) an air ionizer device, and (iii) an airflow device, wherein: (a) the device comprises one or more first openings and a front part comprising one or more second openings: (b) the air ionizer device is configured to generate in an operational mode positively charged particles at a first electrode and negatively charged particles at a second electrode: (c) the airflow device is configured to generate in the operational mode a first airflow entraining the positively charged particles and a second airflow entraining the negatively charged particles: wherein the one or more first openings are configured upstream of the airflow device and the electrodes: wherein a first part of the one or more second openings is configured downstream of the airflow device and the first electrode, and a second part of the one or more second openings is configured downstream of the airflow device and the second electrode; and (d) the device comprises a light emitting area, from which during operation of the light generating device, device light may escape from the device, wherein the front part comprises the light emitting area.

Such a device may provide a more efficient disinfection and may be used in larger areas. Further, the system or ionizer device may allow a relatively easy integration in existing lighting systems. Further, the system or ionizer device may e.g. allow a grid of air treatment devices. This may facilitate a relative even disinfection over rooms, in contrast to disinfection systems that are implemented in (existing) climate control systems. Yet further, the ionizer device may be relatively small. Therefore, the system or ionizer device may also be used in retrofit design of existing devices, especially devices which may be applied in grids, as the air ionizer device may relatively small. With such device both a lighting function and an air treatment function may be provided. Further, such device may be relatively small. Nevertheless, the lighting function may essentially not detrimentally be affected by the disinfection function, whereas the disinfection function may be relatively good. Simulations showed that the different ion streams may propagate relatively far from the device, without substantial detrimental neutralization of the negatively charged particles and the positively charged particles by reaction with each other. Further, such device may have essentially the same function as a normal lighting device not having a function of generating ions, like a normal (retrofit) spot light. The openings in the front part may be relatively small or substantially invisible, and may be fully integrated in the front part. This may also allow a device having a lighting function essentially undisturbed from the presence of the second openings. Further, such device may provide a relatively homogeneous lighting or an essentially state of the art light distribution, even though having the function of also being able to provide ionized air.

As indicated above, the device may comprise a light generating device. The light generating device may comprise one or more light sources.

The term “light source” may in principle relate to any light source known in the art. It may be a conventional (tungsten) light bulb, a low-pressure mercury lamp, a high-pressure mercury lamp, a fluorescent lamp, a LED (light emissive diode). In a specific embodiment, the light source comprises a solid state LED light source (such as a LED or laser diode (or “diode laser”)). The term “light source” may also relate to a plurality of light sources, such as 2-200 (solid state) LED light sources. Hence, the term LED may also refer to a plurality of LEDs. Further, the term “light source” may in embodiments also refer to a so-called chips-on-board (COB) light source. The term “COB” especially refers to LED chips in the form of a semiconductor chip that is neither encased nor connected but directly mounted onto a substrate, such as a printed circuit board (PCB). Hence, a plurality of light semiconductor light source may be configured on the same substrate. In embodiments, a COB is a multi-LED chip configured together as a single lighting module.

The light source has a light escape surface. Referring to conventional light sources such as light bulbs or fluorescent lamps, it may be outer surface of the glass or quartz envelope. For LED's it may for instance be the LED die, or when a resin is applied to the LED die, the outer surface of the resin. In principle, it may also be the terminal end of a fiber. The term escape surface especially relates to that part of the light source, where the light actually leaves or escapes from the light source. The light source is configured to provide a beam of light. This beam of light (thus) escapes from the light exit surface of the light source.

The term “light source” may refer to a semiconductor light-emitting device, such as a light emitting diode (LEDs), a resonant cavity light emitting diode (RCLED), a vertical cavity laser diode (VCSELs), an edge emitting laser, etc. . . . . The term “light source” may also refer to an organic light-emitting diode, such as a passive-matrix (PMOLED) or an active-matrix (AMOLED). In a specific embodiment, the light source comprises a solid-state light source (such as a LED or laser diode). In an embodiment, the light source comprises a LED (light emitting diode). The terms “light source” or “solid state light source” may also refer to a superluminescent diode (SLED).

The term LED may also refer to a plurality of LEDs.

The term “light source” may also relate to a plurality of (essentially identical (or different)) light sources, such as 2-2000 solid state light sources. In embodiments, the light source may comprise one or more micro-optical elements (array of micro lenses) downstream of a single solid-state light source, such as a LED, or downstream of a plurality of solid-state light sources (i.e. e.g. shared by multiple LEDs). In embodiments, the light source may comprise a LED with on-chip optics. In embodiments, the light source comprises a pixelated single LEDs (with or without optics) (offering in embodiments on-chip beam steering).

In embodiments, the light source may be configured to provide primary radiation, which is used as such, such as e.g. a blue light source, like a blue LED, or a green light source, such as a green LED, and a red light source, such as a red LED. Such LEDs, which may not comprise a luminescent material (“phosphor”) may be indicated as direct color LEDs.

In other embodiments, however, the light source may be configured to provide primary radiation and part of the primary radiation is converted into secondary radiation. Secondary radiation may be based on conversion by a luminescent material. The secondary radiation may therefore also be indicated as luminescent material radiation. The luminescent material may in embodiments be comprised by the light source, such as a LED with a luminescent material layer or dome comprising luminescent material. Such LEDs may be indicated as phosphor converted LEDs or PC LEDs (phosphor converted LEDs). In other embodiments, the luminescent material may be configured at some distance (“remote”) from the light source, such as a LED with a luminescent material layer not in physical contact with a die of the LED. Hence, in specific embodiments the light source may be a light source that during operation emits at least light at wavelength selected from the range of 380-470 nm. However, other wavelengths may also be possible. This light may partially be used by the luminescent material.

In embodiments, the light generating device may comprise a luminescent material. In embodiments, the light generating device may comprise a PC LED. In other embodiments, the light generating device may comprise a direct LED (i.e. no phosphor). In embodiments, the light generating device may comprise a laser device, like a laser diode. In embodiments, the light generating device may comprise a superluminescent diode. Hence, in specific embodiments, the light source may be selected from the group of laser diodes and superluminescent diodes. In other embodiments, the light source may comprise an LED.

In specific embodiments, the light generating device may comprise a plurality of different light sources, such as two or more subsets of light sources, with each subset comprising one or more light sources configured to generate light source light having essentially the same spectral power distribution, but wherein light sources of different subsets are configured to generate light source light having different spectral distributions. In such embodiments, a control system may be configured to control the plurality of light sources. In specific embodiments, the control system may control the subsets of light sources individually.

Especially, in embodiments the light generating device comprises a plurality of solid state light sources. Especially, the light generating device is configured to generate (during an operational mode) light generating device light. At least part of this light may escape from the device via the front part (as device light) (see also below). In specific embodiments, in an operational mode, the light generating device may be configured to generate white light. Hence, in embodiment the device may be configured to generate in an operational mode white device light (escaping from the device via the light emitting area). Alternatively or additionally, in another operational mode, the light generating device may be configured to generate colored light. Hence, in embodiment the device may be configured to generate in an operational mode colored device light (escaping from the device via the light emitting area).

The system, or apparatus, or device may execute an action in a “mode” or “operation mode” or “mode of operation” or “operational mode”. Likewise, in a method an action or stage, or step may be executed in a “mode” or “operation mode” or “mode of operation” or “operational mode”. This does not exclude that the system, or apparatus, or device may also be adapted for providing another controlling mode, or a plurality of other controlling modes. Likewise, this may not exclude that before executing the mode and/or after executing the mode one or more other modes may be executed.

However, in embodiments a control system may be available, that is adapted to provide at least the controlling mode. Would other modes be available, the choice of such modes may especially be executed via a user interface, though other options, like executing a mode in dependence of a sensor signal or a (time) scheme, may also be possible. The operation mode may in embodiments also refer to a system, or apparatus, or device, that can only operate in a single operation mode (i.e. “on”, without further tunability).

The light generating device may comprise one or more (solid state) light sources, especially a plurality of solid state light sources. The device may comprise a support for the one or more (solid state) light sources, especially a plurality of solid state light sources. In embodiments, the support may be a PCB, though other supports may also be possible. In embodiments, the one or more (solid state) light sources, especially a plurality of solid state light sources may be configured in a light mixing chamber, which may essentially be closed, and which may comprise a (light transmissive) optical element. The (light transmissive) optical element may define the light emitting area. The light mixing chamber may partly be defined by the support of the one or more (solid state) light sources, especially a plurality of solid state light sources. In embodiments, the support may be reflective for the light source light of the one or more (solid state) light sources, especially a plurality of solid state light sources. Hence, the light mixing chamber may essentially be defined by the support for the one or more (solid state) light sources, the optical element, and optional light chamber (side) walls bridging the support and the optical element. In specific embodiments, the support for the one or more (solid state) light sources may also partly define one or more of the airflow channels. The support for the one or more light sources may also be indicated as “light source support”. The support for the one or more (solid state) light sources may also support other elements.

Further, the device may comprise an ionizer device and an airflow device. In this way, the device may comprise an air treatment device comprising an air ionizer device and an airflow device. The device may comprise one or more ionizer devices and/or one or more airflow devices. In specific embodiments, the devices may comprise a single ionizer device and a single airflow device. In other embodiments, the device comprises up to four ionizer devices, such as one ionizer device or two ionizer devices. With e.g. two ionizer devices, multiple directions of ions flows may even better be controlled. Especially, the air treatment device may comprise a single air ionizer device. However, herein it is not excluded that the air treatment device comprises a plurality of essentially the same and/or a plurality of different ionizer devices. Air ionizers are known in the art.

Note that a single ionizer device may especially comprise at least two electrodes (see further below).

In embodiments, the air ionizer device comprises a set of electrodes. Hence, the air ionizer device may in embodiments have only two electrodes. However, in other embodiments, the air ionizer device may comprise more than two electrodes. For instance, in embodiments the air ionizer device may comprise a plurality of sets of electrodes. Such plurality of sets of electrodes may in embodiments be controlled as if the air ionizer device comprises a set of first electrodes and a set of second electrodes, wherein all first electrodes and all second electrodes are operated in the same way, respectively. Yet, in embodiments the air ionizer device may comprise a plurality of sets of electrodes, wherein two or more sets of electrodes may be controlled individually.

In embodiments, the air ionizer may comprise needles or brushes functioning as ion emitters (i.e. as electrodes). In embodiments, the ion emitters may comprise one or more of tungsten, titanium, steel, and carbon, such as needles or brushes comprising one or more of tungsten, titanium, steel, and carbon. In embodiments, ions may be generated on the basis of the corona effect. In specific embodiments, the electrodes comprise carbon fiber brushes.

Herein, it is distinguished between first electrodes and second electrodes. Hence, whatever set(s) of electrodes is available, one or more electrodes may be indicated as first electrodes and one or more electrodes may be indicated as second electrodes. These electrodes may in embodiments be completely identical, but may in embodiments only differ in the fact that during at least an operational mode the potentials applied to the first electrode(s) and second electrode(s) differ in sign. The value of the potentials may be the same or may differ, but in specific embodiments, at least the sign may differ during at least an operational mode.

In specific embodiments the electrodes may have a mutual electrode distance (d) selected from the range of 5-1500 mm, such as selected from the range of 20-100 mm, like at least 80 mm. In other specific embodiments the electrodes may have a mutual electrode distance (d) selected from the range of 5-150 mm, such as selected from the range of 5-80 mm, like at least 10 mm, such as selected from the range of 10-80 mm. The mutual electrode distance is defined as the shortest distance between tips (extremities) of the electrodes, not taking into account physical barriers.

Especially, in embodiments the air ionizer device may be configured to generate in a first operational mode voltage differences relative to a mutual ground to the set of electrodes. In the first operational mode, positively charged particles may be generated at a first electrode of the set of electrodes, and negatively charged particles may be generated at a second electrode of the set of electrodes. The potentials applied to the first and the second electrode may especially differ in sign, and may also optionally differ in value. However, they may also have the same potential (but different signs).

In embodiments, the positively charged particles may e.g. comprise (organic) molecules that are positively charged. In embodiments, the positively charged particles may e.g. comprise positively charged ions. In embodiments, the negatively charged particles may e.g. comprise molecules that are negatively charged. In (specific) embodiments, the negatively charged particles may e.g. comprise negatively charged ions. In embodiments, the negatively charged particles may e.g. comprise electrons. Hence, in embodiments, the air ionizer device may be configured to generate positive and negative ions. Especially, in embodiments, the air ionizer device may be configured to generate positively charged ions and negatively charged ions. Hence, molecules in air may be ionized by the air ionizer device, thereby generating positively charged ions and/or negatively charged ions. In embodiments, the positively charged ions may be positively charged molecules. In embodiments, the negatively charged ions may be negatively charged molecules.

In general, during operation relatively high voltages may be applied, like selected from the range of 1-10 kV (i.e. in the range of −1 to −10 kV or in the range of 1 to 10 kV), such as selected from the range of 2-8 kV. For instance, the first electrode may have a potential of +4 kV and the second electrode may have a potential of −4 KV, or e.g. the first electrode may have a potential of +3.5 kV and the second electrode may have a potential of −4 kV.

The term “mutual ground” is known to a person skilled in the art, and may in embodiments refer to a defined (coupled) ground to a secondary ground of driver electronics that generates the (high) voltage for the electrodes. It appears desirable to have a defined voltage for generation of the ions. The secondary ground may either be connected to the primary ground via Y-capacitor and safety resistor or via a capacitor only. The secondary ground can be visible (ring or pin) or invisible (internal). For the sake of completeness, it may also be allowed to have this common (mutual) ground on a controlled (DC) voltage level.

Hence, the system, especially the air treatment device (such as in specific embodiments the air ionizer device), may further comprise driver electronics configured to generate the (high) voltage for the first electrode and the second electrode.

Especially, in embodiments the first electrode may at least partly be comprised in a first compartment, functionally coupled to a first part of the one or more second openings (see further below), and the second electrode may at least partly be comprised in a second compartment, functionally coupled to a second part of the one or more second openings (see further below).

For instance, the air ionizer device may be operated at AC, such as in specific embodiments 24 V AC, though other options may also be possible. In general, the air ionizer device may be operated at AC, or at least one electrode at positive potential, with optionally an amplitude on the signal, and at least one electrode at negative potential, with optionally an amplitude on the signal. Even though the air ionizer device may be operated at AC, it is herein also included that during part of the time the potential may be reversed, e.g. for cleaning purposes (see also below at second operational mode). In order not to annihilate its own ions, the time period with opposite signs may either be short, or both the first operational mode and second operational mode may be relatively long. For instance, the first operational mode may last at least 1 minute, such as at least 10 minutes, or even at least an hour, like at least 2 hours.

The terms “first compartment” and “second compartment” may also refer to “first airflow channel” and “second airflow channel”, respectively.

In embodiments, the air ionizer device may be functionally coupled to a single first electrode, at least partly configured in the first airflow channel, and to a single second electrode, at least partly configured in the second airflow channel. In yet other embodiments, the air ionizer device may be functionally coupled to two or more, such as up to four, like two, first electrodes, at least partly configured in a single first airflow channel, and to a single second electrode, at least partly configured in the second airflow channel. In yet other embodiments, the air ionizer device may be functionally coupled to a single first electrode, at least partly configured in the first airflow channel, and to two or more, such as up to four, like two, second electrodes, at least partly configured in a single second airflow channel. In yet other embodiments, the air ionizer device may be functionally coupled to two or more, such as up to four, like two, first electrodes, at least partly configured in a single first airflow channel, and to two or more, such as up to four, like two, second electrodes, at least partly configured in a single second airflow channel. When using more than one electrode in the same channel, the number of ions that may be generated can be higher.

In yet other embodiments, the air ionizer device may be functionally coupled to two or more, such as up to four, like two, first electrodes, at least partly configured in two or more, such as up to four, like two respective first airflow channels, and to two or more, such as up to four, like two, second electrodes, at least partly configured in two or more, such as up to four, like two respective second airflow channel. However, in yet other embodiments, a plurality of air ionizers may be applied, each functionally coupled with at least one airflow channel, such as especially at least two air flow channels (see also below).

In specific embodiments, the air ionizer device, and a control system, may be configured to individually control the two or more first electrodes and/or the two or more second electrodes.

In yet other embodiments, the device may comprise more than two airflow channels. In specific embodiments, the device may comprise selected from the range of a-8*a, such as selected from the range of a-4*a, like selected from the range of a-2*a airflow channels, wherein a is especially two. Hence, in specific embodiments, the device may comprise selected from the range of b-8*b, such as selected from the range of b-4*b, like selected from the range of b-2*b first parts, and selected from the range of b-8*b, such as selected from the range of b-4*b, like selected from the range of b-2*b second parts, wherein b is especially one. The term “a-8*a” and similar terms in this paragraph, refer to a minimum of a and a maximum of 8*a, and all intermediate (integer) values.

In each airflow channel at least part of an electrode may be configured, such as part of a first electrode and part of a second electrode. A single ionizer device may be functionally coupled to all electrodes. In yet other embodiments, the device may comprise two or more ionizer devices, wherein each ionizer device is functionally coupled to one or more, especially two or more electrodes. Hence, the total number of electrodes may in embodiments be configured in subsets, which may be controlled by a single ionizer device (and control system), or by respective ionizer devices (and control system(s)).

As indicated above, the electrodes may be provided by ionization components, such as needles or brushes. Hence, the electrodes may comprise needles or brushes. Especially, the ions may be generated at the tips of such needles or brushes. Therefore, the phrase “at least part of the electrode configured in the airflow channel”, and similar phrases, especially indicate that at least part of the needles or brushes, more especially their tips, are configured in the airflow channels. Likewise, the phrase “downstream of the first electrode” or “upstream of the first electrode”, and similar phrases, especially refer to the needles or brushes, more especially their tips.

The device may further comprise an airflow device. The airflow device may especially comprise a fan, though other options may also be possible. Such fan may produce an air flow in a target direction which may transport ions produced by the air ionizer in the target direction. The term “fan” may refer to any device that can generate a flow, with or without rotating blades. Further, the term “fan” may also refer to a plurality of (individually controlled) fans. Hence, a single airflow device may be used for the two airflow channels. However, also two airflow devices may be used, one for each airflow channel. Also, more than one airflow device may be used for a single airflow channel. When there are more than two airflow channels, the device may comprise one or more airflow devices.

The airflow channels may have a first end, which may especially be defined by the airflow device, and a second end, which may especially be defined by the respective second opening(s).

In embodiments, the volumetric ion flows of the first airflow and the second airflow may be the same. In yet other embodiments, the volumetric ion flows of the first airflow and the second airflow may differ. In embodiments, the volumetric ion flow rate may be defined as the number of ions that pass per volume per unit time, e.g. in ions per cubic meter per second.

In specific embodiments, the airflow device may be functionally coupled to the (afore-mentioned) first compartment and to the (afore-mentioned) second compartment, functionally coupled to first part and second part, respectively, of the one or more second openings (see further below).

In yet other embodiments, the air treatment device may comprise at least two airflow devices, one functionally coupled with the (afore-mentioned) first compartment and another one functionally coupled to the (afore-mentioned) second compartment.

Especially, the air treatment device, may be configured to generate in the first operational mode a set of airflows using the airflow device, wherein a first airflow of the set of airflows entrains the positively charged particles, and wherein a second airflow of the set of airflows entrains the negatively charged particles. To this end, the system or and/or the treatment device may comprise at least two channels and functionally coupled exits. For instance, the ionizer device may comprise two ducts (or airflow channels), wherein the first electrode is at least partly integrated in a first duct, and the second electrode may at least partly be integrated in a second duct. The flow may facilitate that the ions generated close or at the electrodes, are transported away from the ionizer device, such as into a space.

The total ion output may e.g. be at least 100*10⁶/cc, like even at least 200*10⁶/cc. However, larger values, such as at least 200*10⁶/cc, or smaller than values than 100*10⁶/cc may also be possible.

In embodiments, in the first operational mode the first airflow may essentially comprise positively charged particles. Optionally, the first airflow may also comprise negatively charged particles. In embodiments, the ratio of the number of positively charged particles to the number of negatively charged particles in the first airflow (in the first operational mode) may be at least 10, like at least 20, such as at least 50, or even larger, like at least 100, though other ratios may also be possible.

In embodiments, in the first operational mode the second airflow may essentially comprise negatively charged particles. Optionally, the second airflow may also comprise positively charged particles. In embodiments, the ratio of the number of negatively charged particles to the number of positively charged particles in the second airflow (in the first operational mode) may be at least 10, like at least 20, such as at least 50, or even larger, like at least 100, though other ratios may also be possible.

In embodiments, in the second operational mode the first airflow may essentially comprise negatively charged particles. Optionally, the first airflow may also comprise positively charged particles. In embodiments, the ratio of the number of negatively charged particles to the number of positively charged particles in the first airflow in the second operational mode may be at least 10, like at least 20, such as at least 50, or even larger, like at least 100, though other ratios may also be possible.

In embodiments, in the second operational mode the second airflow may essentially comprise positively charged particles. Optionally, the second airflow may also comprise negatively charged particles. In embodiments, the ratio of the number of positively charged particles to the number of negatively charged particles in the second airflow (in the second operational mode) may be at least 10, like at least 10, such as at least 50, or even larger, like at least 100, though other ratios may also be possible.

The first operational mode and the second operational mode may overlap in time at least partly of fully.

In embodiments, during an operational mode essentially only positively charged particles may be generated, and essentially no negatively charged particles, e.g. by applying a potential to only one of the electrodes. Alternatively or additionally, during an operational mode essentially only negatively charged particles may be generated, and essentially no positively charged particles, e.g. by applying a potential to only one of the electrodes. The operational modes may e.g. essentially not overlap in time.

Further, as indicated above, the air ionizer device may be configured to generate in an operational mode positively charged particles at the first electrode and negatively charged particles at the second electrode. However, also the other way around may be executed during an operational mode. And in yet other operational modes, the generation of the positively and negatively charged particles may be executed alternatingly at the same first electrode or the same second electrode.

Especially, the device may have a length from a first end to a second end. The first end and the second end may thus define a total length of the device. Further, the device may comprise a front part and an end part. In between the front part and the end part, there may be a middle part. The front part may comprise the first end. The end part may comprise the second end.

In embodiments, especially the shape of the device may essentially be defined by the front part, the middle part, and the end part. The front part, the middle part, and the end part may in embodiments define an enclosure arrangement. The enclosure arrangement may comprise an envelope (or in specific embodiments be an envelope).

In embodiments, the front part may have a length up to about 25% of the total length of the device, such as in the range of 2-20%, like about 5-20%. In embodiments, the end part may have a length up to about 25% of the total length of the device, such as in the range of 2-20%, like about 5-20%. The middle part may have the remaining length of the total length, such as in the range of about 50-96% of the total length, like in the range of about 60-90%. The device may have a device axis. Especially, the lengths are defined parallel to the device axis. A length axis may coincide with the device axis. Hence, in embodiments the device axis may also be indicated as “longitudinal axis”.

In specific embodiments, the front part may have a first maximum circular equivalent diameter D_{cm,1}, the middle part may have a second maximum circular equivalent diameter D_{cm,2}, and the end part may have a third maximum circular equivalent diameter D_{cm,3}. In specific embodiments, D_{cm,3}≤D_{cm,2}≤D_{cm,1}, but especially at least D_{cm,3}<D_{cm,1}. For instance, in embodiments 1.25*D_{cm,3}<D_{cm,1}<15*D_{cm,3}, such as 1.5*D_{cm,3}<D_{cm,1}<10*D_{cm,3}.

The equivalent circular diameter (or ECD) (or “circular equivalent diameter”) of an (irregularly shaped) two-dimensional shape is the diameter of a circle of equivalent area. For instance, the equivalent circular diameter of a square with side a is 2*a*SQRT(1/π). For a circle, the diameter is the same as the equivalent circular diameter. Would a circle in an xy-plane with a diameter D be distorted to any other shape (in the xy-plane), without changing the area size, than the equivalent circular diameter of that shape would be D.

In specific embodiments, over at least part of the total length, the device tapers in a direction from the first end to the second end. For instance, the middle part may comprise a part that tapers in a direction from the first end to the second end. However, in embodiments also the front part may in embodiments have a part that tapers from the first end to the second end.

In other embodiments, D_{cm,2}≤D_{cm,3}≤D_{cm,1}, but especially at least D_{cm,2}<D_{cm,1}. For instance, in embodiments 1.25*D_{cm,2}<D_{cm,1}<15*D_{cm,2}, such as 1.5*D_{cm,2}<D_{cm,1}<10*D_{cm,2}. For instance, this may be these when the device may provide a substantial spherical shape of the combination of the first part and the second part, with a screw cap, like some E27 type retrofit lamps.

In specific embodiments, the front part may have a part, closer to the first end that tapers in a direction from the second end to the first end, and an adjacent part that tapers from the first end to the second end.

Especially, in embodiments the front part may have essentially circular cross-section (which may especially be defined perpendicular to the device axis) over its entire length. Further, especially in embodiments the middle part may have essentially circular cross-section (which may especially be defined perpendicular to the device axis) over its entire length. Yet further, especially in embodiments the end part may have essentially circular cross-section (which may especially be defined perpendicular to the device axis) over its entire length.

In yet other embodiments, however, the front part may have essentially non-circular cross-section (which may especially be defined perpendicular to the device axis) over its entire length. Further, especially in embodiments the middle part may have essentially non-circular cross-section (which may especially be defined perpendicular to the device axis) over its entire length. Yet further, especially in embodiments the end part may have essentially non-circular cross-section (which may especially be defined perpendicular to the device axis) over its entire length.

In yet other embodiments, two or three of the front parr, the middle part, and the end part may have differently shaped cross-sections, like e.g. one may have a square cross-section and another one may have a circular cross-section.

When viewed along the device axis, the front part may have a largest cross-sectional area (perpendicular to the device axis) of in embodiments at least 2 cm², such as selected from the range of 2-400 cm², like selected from the range of 3-100 cm², such as up to about 50 cm².

The device may comprise one or more first openings and one or more second openings. The one or more first openings may have the function of an inlet. The one or more second openings, or at least part(s) thereof may have the function of an exhaust (for the ionized particles).

Especially, the one or more first openings are configured further away from the first end than the one or more second openings. Likewise, the one or more first openings are configured relatively closer to the second end than the one or more second openings.

For instance, in embodiments the one or more first openings are comprised by the middle part and the one or more second openings are comprised by the front part. Especially, the one or more second openings are comprised by the front part and the one or more first openings are comprised by one or more of the front part, the middle part and the end part, especially by one or more of the middle part and the end part. In specific embodiments, the one or more first openings are comprised by the middle part.

In this way, air introduced via the first openings further away from the first end (than the second openings) may escape the one or more second openings closer to the first end (than the one or more first openings). In this way, light and/or ion flows may escape from the front part (see also below) or light may escape from the front part and ion flows may escape from the middle part.

Therefore, the device comprises one or more first openings and a front part comprising the one or more second openings. For further embodiments of the openings, see also below. Especially, in embodiments the device comprises at least two first openings.

The airflow device may be configured to create an internal airflow from the one or more first openings to the one or more second openings. Hence, the airflow device may suck air (via the one or more first openings) into the device and exhaust from the device at the one or more second openings. Especially, however, the one or more first openings are configured upstream of the airflow device, whereas downstream of the airflow device there are at least two channels (“airflow channels”): a first channel in the direction of a first part of the one or more second openings with at least part of the first electrode configured in the first channel, and a second channel in the direction of a second part of the one or more second openings with at least part of the second electrode configured in the second channel. In this way, the positively charged particles and the negatively charged particles may essentially be kept separated (within the device).

The terms “upstream” and “downstream”, such as in the context of a flow of a fluid, like a liquid or a gas, may especially relate to an arrangement of items or features relative to the flow of the fluid coming from a source of the fluid (which may here be the external directly upstream of the one or more first openings), wherein relative to a first position within (or along) a flow of the fluid from the source of the fluid, a second position in (or along) the flow of fluid closer to the source of the fluid (than the first position) is “upstream”, and a third position within (or along) the flow of the fluid further away from the source of the fluid (than the first position) is “downstream”.

Hence, the airflow device may be configured to generate in the operational mode a first airflow entraining the positively charged particles and a second airflow entraining the negatively charged particles. Especially, the one or more first openings are configured upstream of the airflow device and the electrodes. In embodiments, a first part of the one or more second openings may be configured downstream of the airflow device and the first electrode, and a second part of the one or more second openings may be configured downstream of the airflow device and the second electrode.

Especially, in embodiments a shortest distance between a first opening and a second opening, taking into account physical barriers, may be selected from the range of 5-1500 mm, such as selected from the range of 20-100 mm, like at least 40 mm. Note that air sucked into the device via a first opening may in embodiments essentially only escape from the device via a second opening. Hence, in embodiments the one or more first openings and the one or more second openings may be configured in a gas flow system, including the airflow device and including the airflow channels downstream of the airflow device.

Further, the airflow device is especially functionally coupled with the one or more first openings. Hence, essentially in embodiments all air sucked into the device via the one or more first openings may escape from the device only via the airflow device (and via the one or more second openings).

In specific embodiments, there may be a single second opening, of which a first part may essentially be used for the exhaust of the positively charged particles, and of which a second part may essentially be used for the exhaust of the negatively charged particles. Especially, the first part and the second part may be configured such, that during operation there is substantially no overlapping airflow within the shared opening. Hence, especially separate second openings are used as exhaust for the first airflow and the second airflow. Therefore, a first part of the one or more second openings may be configured as exhaust for the first airflow and a second part of the one or more second openings may be configured as exhaust for the second airflow.

The term “first part” may in this context especially refer to one or more second openings. The term “second part” may in this context especially refer to one or more other second openings.

In specific embodiments, the first part comprises a single second opening. This may allow limiting the possible number of blocking elements. In other embodiments, the second part comprises a single second opening (other than the single second opening comprised by the first part). Also, this may allow limiting the possible number of blocking elements.

In specific embodiments, the first part comprises a plurality of second openings, like 2-4. This may still allow limiting the possible number of blocking elements but may also allow a further control of the flow characteristics, like direction (of the airflow). In other embodiments, the second part comprises a plurality of second opening, like 2-4 (other than the single second opening comprised by the first part). This may still allow limiting the possible number of blocking elements but may also allow a further control of the flow characteristics like direction (of the airflow).

In specific embodiments, the first part comprises a plurality of second openings, like 5-20. This may allow control of the flow characteristics, like direction (of the airflow). In other embodiments, the second part comprises a plurality of second opening, like 5-20 (other than the single second opening comprised by the first part). This may still allow control of the flow characteristics like direction (of the airflow).

In embodiments, a plurality (like 5-20) of second openings comprised by the first part, may be configured in a (first) array, wherein the openings in the (first) array may be spatially separated by fins (see also below). In embodiments, a plurality (like 5-20) of second openings comprised by the second part, may be configured in a (second) array, wherein the openings in the (second) array may be spatially separated by fins (see also below).

When there are a plurality of second openings, in embodiments two or more second openings may be configured parallel and/or two or more second openings may be configured in series.

Especially, the first part and the second part are spatially separated. The shortest distance between the first part and the second part, taking into account physical barriers, may be about 5-150 mm, such as selected from the range of 5-80 mm, like at least 10 mm. In specific embodiments, the shortest distance between the first part and the second part, taking into account physical barriers, may at least be about 15 mm.

As indicated above, the front part may comprise the one or more second openings. Also, the light of the light generating device may escape from the front part.

As indicated above, the device comprises a light generating device. In embodiments, the light generating device may be configured to generate light generating device light. At least part of the light generating device light may escape from the device via the front part. The light that escapes from the device may be indicated as device light and may comprise at least part of the light generating device light.

In specific embodiments, the spectral properties of the device light may be controllable (by a control system): see further also below:

In an operational mode, the light generating device may be configured to provide white device light. Optionally, in embodiments in an (other) operational mode the light generating device may be configured to provide colored device light.

The term “white light” herein, is known to the person skilled in the art. It especially relates to light having a correlated color temperature (CCT) between about 1800 K and 20000 K, such as between 2000 and 20000 K, especially 2700-20000 K, for general lighting especially in the range of about 2700 K and 6500 K. In embodiments, for backlighting purposes the correlated color temperature (CCT) may especially be in the range of about 7000 K and 20000 K. Yet further, in embodiments the correlated color temperature (CCT) is especially within about 15 SDCM (standard deviation of color matching) from the BBL (black body locus), especially within about 10 SDCM from the BBL, even more especially within about 5 SDCM from the BBL.

The device may comprise a light emitting area, from which during operation of the light generating device, device light may escape from the device. Especially, the front part comprises the light emitting area. In embodiments, the light emitting area may be the surface of a lens, or the surface of a diffuser, or the surface of a light transmissive window (such as e.g. of glass, quartz, or polymeric material), or the surface of a homogenizer body, or the surface of a reflector body (e.g. based on total internal reflection), or the surface of a light conversion element, or any other (usable) optical element. Especially, the light emitting area may thus be an end window. Would there be no optical element downstream of the one or more light sources, the light emitting surface(s) of the one or more light sources may provide the light emitting area. Especially, however an optical element may be configured downstream of the one or more light sources. note that a light transparent window which may have essentially no impact on the spectral power distribution or beam shape may also be considered an optical element. Such optical element may e.g. be available for protection of the one or more (solid state) light sources.

Especially, in embodiments the light emitting area may have an essentially circular cross-section (which may especially be defined perpendicular to the device axis). Note that the light emitting area may in embodiments be essentially planar. However, in other embodiments the light emitting area may b ID curved, or 2D curved, especially 2D curved. As can be derived from the above, the light emitting area may have the shape of a spherical segment, or of an in two perpendicular directions elongated spherical segment (like a frisbee).

Especially, in embodiments the optical element may have an essentially circular cross-section (which may especially be defined perpendicular to the device axis).

The optical element may have an outer surface area (which may essentially be the light emitting area), of at least 2 cm², such as selected from the range of 2-400 cm², like selected from the range of 3-100 cm², such as up to about 50 cm².

The light emitting area may be defined by the one or more light sources, but may especially be configured by an optical element configured by the one or more light sources. The optical element may (thus) be configured downstream of the one or more light sources, especially solid state light sources. The optical element may be comprised by a front element, or may be a front element. The front element may especially be comprised by the front part.

In embodiments, the optical element may comprise a polymeric material, like a PC window or a PMMA window. In other embodiments, the optical element may comprise a glass or quart window.

The terms “upstream” and “downstream”, such as in the context of propagation of light, may especially relate to an arrangement of items or features relative to the propagation of the light from a light generating means (here the especially the one or more light sources, especially solid state light source), wherein relative to a first position within a beam of light from the light generating means, a second position in the beam of light closer to the light generating means (than the first position) is “upstream”, and a third position within the beam of light further away from the light generating means (than the first position) is “downstream”.

Hence, the device may in embodiments comprise one or more light sources, especially solid state light sources, and a front element, wherein the optical element is configured downstream of the one or more (solid state light sources). In specific embodiments, the front element comprises an optical element (comprising the light emitting area). Yet, in further embodiments the first part (of the one or more second openings) and the second part (of the one or more second openings) may at least partly enclose the optical element. However, other embodiments may also be possible.

The front element may have a disc-like shape. However, the front element may also have a kind of lid-like shape. The front element may have curved edges. The front element may have edges that also extend in the direction of the second end of the device, thereby providing a part of an enclosure. The front element may have the shape of a top part of a bulb, like retro incandescent lamps. The front element may have the shape of at top part of a flattened bulb, like (retro) reflector lamps. For instance, the front element may have the shape of the cover plate as described in EP0516231A2, which is herein incorporated by reference. However, other shapes may be possible as well.

The front element may in embodiments define a substantial part of the front part.

In specific embodiments, the optical element may comprise a translucent window. Hence, in embodiments the optical element may comprise a diffuser. Part of the light of the one or more (solid state) light sources may be reflected/scattered (back) by the translucent optical element (such as translucent window). The reflectivity of the translucent window or diffuser may be in the range from 20 to 60% from the light of the one or more (solid state) light sources. In this way, e.g. a light mixing chamber may be provided.

In other embodiments, the optical element may comprise a transparent material, such as a collimator body, or a lens. In yet other embodiments, the optical element may comprise a transparent material, having essentially no further optical function than allowing transmission of the light of the (solid state) light source(s). Combinations of optical elements may also be applied. See further also above. The most downstream configured optical element of the device may define the light emitting area.

As indicated above, the front part may comprise the light emitting area. Further, as indicated above, the front part may comprise the one or more second openings. Especially, in embodiments the front part may comprise the first part comprising one or more second openings, and the second part comprising one or more (other) second openings. Further as indicated above, the front part may comprise an optical element. Hence, in specific embodiments the optical element may at least partly be surrounded by the first part and the second part. In yet more specific embodiments, the first part may comprise 1-4 second openings, and the second part comprises 1-4 second openings. However, as indicated above, larger number of openings may also be possible. Especially, in embodiments the optical element is may be completely surrounded by the first part and the second part.

For instance, in embodiments the front element may comprise a central part and a peripheral part. Especially, in embodiments the central part may have an essentially circular cross-section (especially defined perpendicular to the device axis). In embodiments, the peripheral part may have an essentially circular cross-section (especially defined perpendicular to the device axis), such as ring shape.

Hence, in embodiments the central part may comprise the optical element, wherein the peripheral part comprises the first part and the second part.

As indicated above, in embodiments the first part may comprise 1-4 second openings, and the second part may comprise 1-4 second openings. In yet further specific embodiments, the front part may have an essentially circular cross-section. The circle may be divided in a first circle section comprising the first part of the one or more second openings and a second circle section comprising the second part of the one or more second openings. Especially, in embodiments there may be a third circle section not comprising second openings. In embodiments, the first circle section and the second circle section are separated by the third circle section. The third circle section may comprise one or more third circle sections. In embodiments, the total third circle section, which may in embodiments comprise two or more (smaller) third circle sections (with a first and/or a second circle section in between two third circle sections), may have an angle of at least 90°, even more especially at least 135°, such as in specific embodiments at least about 180°. Hence, assuming in embodiments a single first circle section, and oppositely configured thereof a second circle section, these two section may be separated by two third circle sections. The latter two third circle section may have angles of especially at least 45°, which may thus provide total third circle section of at least 90°. The circle sections may herein also be indicated as “circular sectors”.

Note that in specific embodiments there may be a ring of one or more, especially a plurality of second openings. In such embodiments, there may be no third circle section.

In specific embodiments, the front element may comprise a monolithic element. Even more especially, the front element may be a monolithic element. For instance, a polymeric element of light transmissive material may comprise a central part that is provided with scattering elements, and a peripheral part, which may optionally be provided with scattering elements, but which may at least comprise the first part and the second part. the front element may e.g. be provided by one or multiple component injection molding, or by (die) casting, 3D printing, etc. In embodiments, the front element may comprise a coating or the front element may be provided with a coating.

In other embodiments, the front element may comprise two or more parts that form an assembly.

In embodiments, the front part may be a monolithic element. In other embodiments, the front part may comprise two or more parts.

The front part may comprise the front element. The front part may in embodiments comprise part of the enclosure arrangement (see also below).

As indicated above, in embodiments the first part and/or the second part may at least partly enclose the optical element.

In specific embodiments when viewed from the front, a viewer may perceive the first part and/or the second part of the second openings. Especially, however, the viewer may not perceive the first openings.

In other embodiments, however, when viewed from the front, a viewer may perceive only part of the first part and/or the second part of the second openings (and the viewer may not perceive the first openings). This may e.g. be the case when the second openings are partly configured in a part of the device that tapers in a direction from the first end to the second end.

In yet other embodiments, however, when viewed from the front, a viewer may not perceive the first part and/or the second part of the second openings (and the viewer may not perceive the first openings). This may e.g. be the case when the second openings are configured in a part of the device that tapers in a direction from the first end to the second end. For instance, when the front element (see also above) may hide the second openings for the viewer (viewed from the front).

Hence, in embodiments at least part of (the first part and/or the second part of) the second openings may be configured behind an edge of the front part, such as an edge of the front element.

In embodiments, the second openings may be curved and may be configured at least partly around the device axis. The second openings may have shortest distances, not taking into account physical barriers, to the device axis selected from the range of 5-150 mm, such as selected from the range of 5-80 mm, like at least 10 mm. Hence, the second openings may be radially arranged. In other embodiments, the second openings may be tangentially arranged.

Relative to the device axis the one or more second openings may surround the device axis over 360°. In other embodiments, however, the one or more second openings may surround the device axis over less than 360°, such as selected from the range of 10-270°, like in embodiments selected from the range of 20-180°. Note that the one or more second openings partially surrounding the device axis may be provided by different openings at the same radius. For instance, two second openings may surround the device axis over 90°, which surrounding angle may be provided by the two second openings, oppositely arranged relative to the device axis and each surrounding the device axis over an angle of 45°.

Referring to the first part of the second openings and the second part of the second openings, in embodiments may surround the device axis over 360°. In other embodiments, however, the first and second part of the one or more second openings may surround the device axis over less than 360°, such as selected from the range of 10-270°, like in embodiments selected from the range of 20-180°. Note that the first and second part of the one or more second openings partially surrounding the device axis may be provided by different spatially separated first part and second part of the one or more second openings, though at the same radius. For instance, the first part and the second part may together surround the device axis over 90°, which surrounding angle may be provided by the two parts, oppositely arranged relative to the device axis and each of the first part of the second openings and the second part of the second openings surrounding the device axis over an angle of 45°.

A second opening of the one or more second openings may have one or two rounded (end) edges. A second opening of the one or more second openings may have one or two slanted end edges (which may optionally also be rounded). When having a slanted (end) edge, the end edge may not be in the same plane as the device axis. Slanted (end) edges may contribute to a desired flow direction. Slanted angles are discussed below in relation to fins. Slanted end edges may provide second openings having kind of (curved) rhomboid cross-sectional shape.

The above described embodiments do not necessarily limit the position, shape, size and number of the one or more second openings comprised by the first part and the one or more second openings comprised by the second part. It is even not excluded that one or more second openings comprised by the first part and the one or more second openings comprised by the second part may differ in one or more e.g. shape and size.

The airflow may leave the device in several ways, which may be defined by the device, especially e.g. flow channels (“airflow channels”) of the device.

Herein, the device is especially described in relation to two airflows, one entraining positive particles and one entraining negative particles. As can be derived from the above, it may also be possible that the device is configured during an operational mode to generate n sets of (each) a first airflow and a second airflow. The number of n may e.g. be selected from the range of 1-8, such as 1-4, like 1-2. For the sake of understanding, herein embodiments of the device is especially described in relation to a single set (n=1) of a first airflow and a second airflow. For instance, this may thus also imply a single set of a first part of the second openings and a second part of the second openings. Notwithstanding that the device is especially described in relation to two airflows, one entraining positive particles and one entraining negative particles, it may be that in operational modes only one of the flows is provided. Hence, the device herein is especially described in relation to two airflows, one entraining positive particles and one entraining negative particles, which will in embodiments be provided at the same time, but may in other embodiments be provided at different times, or in non-overlapping or partly overlapping pulse periods.

Especially, in embodiments the airflows with the positive and negative particles, respectively, may leave the device at spatially separated positions as this may reduce the chance on an early recombination of the positively and negatively charged particles (see also above).

Especially, during an operational mode wherein at the same time (or optionally within about 5 seconds of each other) the first airflow and the second airflow are provided from different second parts, the shortest distance (d1) between those second parts, taking into account physical barriers, may be about 5-150 mm, such as selected from the range of 5-80 mm, like at least 10 mm.

Especially, the first airflow has a first direction and the second airflow has a second direction. It appears useful to separate the positively charged particles and the negatively charged particles. For that reason, the first electrode and the second electrode may be configured in different compartments, may receive different flows, leading to airflows with ions with opposite signs, propagating in different directions and/or at least leaving the device at spatially separated positions.

Assuming two airflows, the device may allow several options how the airflows leave the device.

For instance, in embodiments the airflow directions may be directed essentially parallel and essentially parallel to the device axis. Such solution may be relatively easy, but may also have a higher chance on recombination than some other solutions described herein. For instance, in other embodiments the airflow directions may be directed essentially parallel but essentially not parallel to the device axis. This may include design choices which facilitate such airflow directions. Also, such solution may also have a higher chance on recombination than some other solutions described herein. For instance, in yet other embodiments the airflow directions may be directed under an angle larger than 0° and (equal to or) smaller than 180° but in a single plane parallel to the device axis. Such solution may be relatively easy, and may have a lower chance on recombination than the other solutions described earlier in this paragraph. For instance, in yet further embodiments the airflow directions may be directed under an angle larger than 0° and (equal to or) smaller than 180°, but not in a single plane parallel to the device axis. This may include design choices which facilitate such airflow directions, and may have a lower chance on recombination than some of the other solutions described earlier in this paragraph, and may even have the lowest chance on recombination.

Hence, referring to the device axis, the two airflow directions may be parallel to the device axis or may not parallel to the device axis. Further, the two airflow directions may be in the same (straight) plane, or may not be in the same (straight) plane.

With the present invention a (kind of) swirl of the airflow may be created internally in the device. Especially, once the airflow is outside of the device, the airflows may have been provided with directions such that the positive particle entraining airflow and negative particle entraining airflow diverge from each other, and may thus in embodiments essentially not be parallel and not converge.

Here below, some embodiments are described.

In embodiments, the first airflow has a first direction having a first angle (α1) relative to the device axis (A1), and the second airflow has a second direction having a second angle (α2) relative to the device axis (A1). Especially, the first angle (α1) and the second angle (α2) are independently selected from the range of 0-180°. However, in specific embodiments one of the first angle and the second angle may be selected from essentially 180°, and the other of the first angle and the second angle may be selected from essentially 0°, for instance in specific embodiments for a pendant application. In such embodiments, the respective airflows may be directed essentially anti-parallel and in a plane essentially parallel to the device axis.

In specific embodiments, the first angle (α1) and the second angle (α2) may independently be selected from the range of 0-150°, such as from the range of 0-135°. In specific embodiments, one of the first angle (α1) and the second angle (α2) may be at least 90° and the other may be at maximum 90°, with a mutual angle of at least 45°, such as especially at least 90°. In specific embodiments, this may be used for a pendant application. Note that would—by way of example—the first angle (α1) and the second angle (α2) both be 135°, then the mutual angle may be 270° (which is effectively 90°), like in the embodiment when the first angle (α1) and the second angle (α2) would both be 45°. However, herein an angle of 0° of an airflow direction may refer to a direction parallel to a direction from the second end to the first end, and an angle of 180° of an airflow direction may refer to a direction anti-parallel to a direction from the second end to the first end.

Especially, however, in embodiments the first angle (α1) and the second angle (α2) may independently selected from the range of 0-90°. Hence, in embodiments the first angle and the second angle may be the same, and in other embodiments, the angles may be different. The angles may be defined by one or more of the second openings (or their shapes) and the upstream shape of the airflow channels, upstream of the second openings.

The mutual angle between the airflows may in specific embodiments thus be 180° and in other specific embodiments 0°. Especially, however, both the first angle and the second angle may be larger than 0° but smaller than 90°. For instance, the first angle and the second angle may be at least 2.5°, such as at least about 4°. Hence, in specific embodiments the first direction and the second direction have a mutual angle (am) selected from the range of 5°-180°. Especially, the mutual angle may be smaller than about 165°, such as selected from the range of 5-165°, like 5-150°.

However, in other embodiments, the mutual angle may be selected from the range of 135-180°. Even, in specific embodiments the mutual angle may be selected from the range of 180-360°. In yet other embodiments, the mutual angle may be selected from the range of 5-90°. Especially, however, the mutual angle is larger than 0° and smaller than 360°, like equal to or smaller than 270°, such as in embodiments equal to or smaller than 180°.

As indicated above, in embodiments the first direction and the second direction may not be configured in a single (straight) plane. Hence, even though in embodiments the two airflows may escape from the device at positions which are in the same plane as the device axis, the directions of the two airflows may not be in the same plane. This may further reduce the chance on annihilation of the charged particles by reaction of positively and negatively charged particles. Further, in this way the airflows may be provided with a kind of twisted directions.

Hence, in embodiments the first direction and the second direction are selected not to be in a single (straight) plane. Amongst others, this may be obtained with the shape of the flow channel and/or with the shape of the second openings (including optional fin: see further below).

To obtain two airflows that may escape at different positions and which have directions that are not in the same plane, even though relative to device axis the positions where the airflows leave the device may be opposite, may in embodiments be due to one or more of (i) the shape of the second openings and (ii) the upstream shape of the airflow channels, upstream of the second openings.

The direction of the airflow may be the main direction directly downstream of the respective first part or second part. This direction may be defined by the direction in which the airflow; especially the volumetric flow rate of the air, is highest. On the basis of the shape of the airflow channels, the relative volumetric flow rates for the first airflow and the second airflow may be determined, when the ratio of the volumetric flow rate provide to the airflow channels is known. For instance, when they share the same airflow device, and are symmetrically configured, the ratio may be 1. For instance, the airflow may be determined with a device suitable for measuring an airflow, like e.g. a (handheld) air velocity meter as known in the art.

For instance, in embodiments the shape of the outlets may be such that there is substantially no mirror plane containing the device axis. For instance, in specific embodiments fins may be used, that even though configured opposite to each other relative to the device axis, are not mirror images of each other in a mirror plane containing the device axis.

In specific embodiments, the openings may comprise fins or may be defined by fins which may not be in a same plane as the device axis (A1) and which may be non-perpendicular to a plane perpendicular to the device axis. This may apply to both the first part of the second openings and the second part of the second openings. Hence, in specific embodiments one or more of the following applies: (a) the first part comprises at least one second opening at least partly defined by fins, which are not in a same plane as the device axis (A1) and which are non-perpendicular to a plane perpendicular to the device axis (A1), and (b) the second part comprises at least one second opening at least partly defined by fins, which are not in a same plane as the device axis (A1) and which are non-perpendicular to a plane perpendicular to the device axis (A1). In this way, e.g. the essentially most downstream configured elements may determine the way in which the first airflow and/or the second airflow may leave the device. Hence, for instance one or more fins may be applied to provide the first airflow and the second airflow with directions which are not configured in the same plane (and not in a plane containing the device axis), while nevertheless the first airflow and second airflow may leave the device at opposite positions relative to the device axis.

One or more of the fins, such as in embodiments a majority of the fins, may define a plane having an angle with the device axis selected from the range of equal to 0°, which may be parallel to the device axis, up to nearly 90°, which may be close to perpendicular to the device axis. The angle the plane through the fins makes with device axis may also be indicated as slant angle (see also above in relation to the end edges of the second openings). Especially, the slant edge may be selected from the range of 7.5-75°, which may e.g. allow a mutual angle of the directions of the airflow selected from the range of 15-150°. However, other slant angles may also be possible. Further two or more fins have different slant angles.

Air forced through an airflow channel may obtain a direction due to the presence of one or more fins and/or one or more slanted end edges of the second opening(s).

In embodiments, one or more second opening may at least partly be defined by one or more fins. In yet other embodiments, wherein the device comprises a plurality of second openings, a first part of the second openings may at least partly be defined by fins, like e.g. a series of four openings partly defined by three fins, and/or a second part of the second openings may at least partly be defined by fins, like e.g. a series of four openings partly defined by three fins. Other embodiments, however, may also be possible.

An airflow channel may comprise one or more second openings. In embodiments, when an airflow channel comprises two or more openings, between adjacent openings, a fin may be configured (especially having a non-zero slant angle). The fin(s) may have a wing shape. Hence, in embodiments the fins may have aerodynamical shapes. In this way, impact on the airflow may be minimized and/or loss of charged particles may be minimized. In specific embodiments, the first part of the one or more second openings or the second part of the one or more second openings may (each independently) comprise one or more, especially two or more, wing-shaped fins.

Hence, in embodiments the first airflow channel may comprise two or more second openings, with fins between adjacent second openings, with the fins especially having a non-zero slant angle. Hence, the first part of the one or more second openings may comprise two or more second openings. Alternatively or additionally, in embodiments the second airflow channel may comprise two or more second openings, with fins between adjacent second openings, with the fins especially having a non-zero slant angle. Hence, the second part of the one or more second openings may comprise two or more second openings.

In other embodiments, however, the first part of the one or more second openings may comprise a single second opening, or 2-4 second openings without fins between adjacent openings, but optionally (second openings) with one or more slanted end edges. Alternatively or additionally, the second part of the one or more second openings may comprise a single second opening, or 2-4 second openings without fins between adjacent openings, but optionally (second openings) with one or more slanted end edges.

Not only the essentially most downstream configured elements may create airflows with directions not in the same plane, alternatively or additionally this may be facilitated with elements upstream of the first part and/or second part of the second openings. For instance, elements configured downstream of the airflow device but upstream of the first part and/or second part of the second openings may direct the respective airflow in such a way, that it leaves the respective part, even in embodiments without using fins which are not in the same plane as the device axis. For instance, this may be obtained with curved channel elements or with other (flow influencing) elements. Such elements may influence the direction of the respective airflow within the device but also effectively at least partly define the direction of the airflow leaving the device.

Hence, downstream of the airflow device, there may be two airflow channels. A first airflow channel may be configured to provide an airflow to the first part of the second openings and a second airflow channel may be configured to provide an airflow to the second part of the second openings. Within the first airflow channel, at least part of the first electrode may be configured, and within the second airflow channel, at least part of the first electrode may be configured. In specific embodiments, there are only two airflow channels, which are configured essentially symmetrical relative to the device axis.

Especially, in embodiments at least part of the first airflow channel, even more especially at least a part of the first airflow channel downstream of the first electrode, and especially essentially the entire part downstream of the first electrode, thus including the first part of the second openings, may be defined by channel walls, which when (virtually) elongated along a channel axis of such channel part downstream of the first electrode may not intersect with the device axis.

Likewise, in embodiments at least part of the second airflow channel, even more especially at least a part of the second airflow channel downstream of the second electrode, and especially essentially the entire part downstream of the second electrode, thus including the second part of the second openings, may be defined by channel walls, which when elongated along a channel axis of such channel part downstream of the second electrode may not intersect with the device axis.

As indicated above, the airflow channels may at least partly be defined by curved channel elements or with other (flow influencing) elements. A flow influencing element may e.g. comprise a bulkhead, a partition, a vane, etc. Hence, in embodiments one or more of the following may apply: (a) a first airflow channel configured between the airflow device and the first electrode comprises a curved shape, and (b) a second airflow channel configured between the airflow device and the second electrode comprises a curved shape.

Further, the airflow channels may be defined by a housing. Yet further, the airflow channels may be defined by a support element. Such support element may e.g. be used to support the electrodes. Such support element may in embodiments also comprise or support one or more flow influencing elements.

Hence, in embodiments the device may further comprise a support element, configured to support the first electrode and the second electrode, wherein in further specific embodiments the support element at least partly defines one or more of the first airflow channel and the second airflow channel. Hence, the support may comprise one or more features like extensions or bends or openings, etc., which may define the flow channel and/or which may have the function of flow influencing element. The support element may also be indicated in embodiments as “bracket”.

Especially, the support element is configured (at least partly) downstream of the flow device. Hence, the support element may comprise an opening at least partly defining the respective flow channel. Alternatively or additionally, the support element together with an enclosure arrangement may define an opening at least partly defining the respective flow channel.

In embodiments, the support element may comprise at least one curved channel elements and/or at least one other flow influencing elements per airflow channel (see further also below).

Yet further, the airflow channels may partly be defined by the support for the one or more (solid state) light sources, such as a PCB.

As indicated above, the device may comprise an enclosure arrangement. The enclosure arrangement may especially comprise a housing. The enclosure arrangement may comprise the middle part. The enclosure arrangement may also comprise the front part and/or the end part. The enclosure arrangement may in embodiments be a monolithic body. In other embodiments, the enclosure arrangement may comprise an assembly of two or more parts.

The enclosure arrangement may comprise a housing with an opening, which may essentially be closed by the front part, such as via a screw connection or a click connection or other connections known in the art. The enclosure arrangement may comprise a housing with an opening, which may essentially be closed by the front element, such as via a screw connection or a click connection or other connections known in the art.

Further, enclosure arrangement, such as in specific embodiments the housing, may also comprise or support one or more flow influencing elements (see also above). Hence, in embodiments the support element may comprise or support one or more flow influencing elements, such as vanes, and the enclosure arrangement may not comprise or support such flow influencing elements. Alternatively, in embodiments the housing may comprise or support such flow influencing elements, such as vanes, and the support element may not comprise or support such flow influencing elements. In vet other embodiments, both the housing and the support element may comprise or support flow influencing elements.

The enclosure arrangement may host the ionizer device, the airflow device, and the light generating device. Further, the enclosure may host electronics.

The enclosure element may enclose the control system, configured to control one or more of the ionizer device, the airflow device, and the light generating device.

Hence, in embodiments the device may comprise an enclosure arrangement, wherein the enclosure arrangement at least partly encloses the light generating device, the air ionizer device, and the airflow device: wherein the device comprises a first end and a second end: wherein the light emitting area is configured closer to the first end than to the second end: wherein the enclosure arrangement comprises the one or more first openings configured between the first end and the second end: wherein the device comprises electrical connectors and electronics, wherein the electronics are at least partly enclosed by the enclosure arrangement, wherein the electronics are functionally coupled to the light generating device, the air ionizer device, the airflow device, and the electrical connectors, and wherein the electrical connectors are configured closer to the second end than the first end. Hence, in embodiments an electrical connector may define the second end. In embodiments, the light emitting area may define the first end.

The electrical connector which may e.g. be functionally connected to the mains during operation of the device. The electrical connectors may in embodiments be defined by an Edison screw cap, such as an A27 screw cap. This may allow an easy implementation in existing lighting systems. Hence, in embodiments the device may comprise an Edison screw cap (Edison screw base) or other type of cap (such as a bayonet base) for functional coupling to a lamp holder and source of electricity.

The cap may in embodiments essentially define the end part. Hence, in embodiments a housing may at one end be closed by the cap and at another end be closed by the front part or by the front element.

As indicated above, the airflow channels may comprise flow inducing elements, or may at least partly be defined by flow inducing elements. In embodiments, the enclosure arrangement may comprise one or more flow inducing elements, which are configured at least partly in the first airflow channel or the second airflow channel.

Hence, one or more of the enclosure arrangement and the support element may comprises one or more flow influencing elements. Especially, the one or more flow influencing elements may at least partly be configured in at least one of the first airflow channel and the second airflow channel. Both types of flow influencing elements may direct the airflow. Flow influencing elements on the support may be easier to make than flow influencing elements on the enclosure arrangement. An advantage of positioning the vanes on the housing (or other parts (like a ventilator holder)) can be ease and cost of manufacturability, flexibility of design options and options for miniaturization, etc.

As indicated above, the front part may comprise the front element. The front part may in embodiments comprise part of the enclosure arrangement or the enclosure arrangement may define at least part of the front element. In embodiments, the enclosure arrangement may substantially be defined by the middle part. In embodiments, the middle part of the enclosure arrangement is a single monolithic body.

The external part of the front part, which may especially comprise at least the light emitting area and in embodiments at least part of the second openings, more especially the entire second openings, may in embodiments be a monolithic body. In other embodiments, it may comprise two or more elements, like a ring shaped element and the front element. The front part may be curved, and may in embodiments comprise the shape of the broadest part of a (retrofit) spotlight.

The front element may in embodiments have an essentially homogeneous light transparent part, especially essentially without obstacles in the optical path. The front part and the (remainder of the) enclosure arrangement may provide a safe interface to the external of the device.

In other embodiments, the device may comprise a luminaire or be a luminaire.

Referring to the air ionizer device and the airflow device, from the second end to the first end, the configuration may be the airflow device closer to the second end than the ionizer device, and the ionizer device closer to the first end than the airflow device.

Referring to the air ionizer device, the support element, and the airflow device, from the second end to the first end, the configuration may be the airflow device closer to the second end than the ionizer device, and the ionizer device closer to the first end than the airflow device, with the support element configured in between.

Referring to the airflow device, the support element and the light generating device, from the second end to the first end, the configuration may be the airflow device closer to the second end than the light generating device, and the support element configured in between.

The ionizer device may be located essentially anywhere in the device, like in the front part, the middle part, or the end part. Hence, in embodiments the ionizer device may be located both in the front of the lamp (near to the light generating device) or in the back of the lamp (e.g. close to an end cap).

An LED driver may be located essentially anywhere in the device, like in the front part, the middle part, or the end part. Hence, in embodiments the LED driver may be located both in the front of the lamp (near to the light generating device) or in the back of the lamp (e.g. close to an end cap).

Likewise, in embodiments electronics to control the airflow device may be located essentially anywhere in the device, like in the front part, the middle part, or the end part. Hence, in embodiments the electronics to control the airflow device may be located both in the front of the lamp (near to the light generating device) or in the back of the lamp (e.g. close to an end cap).

Likewise, this may in embodiments apply to (possible) other electronics.

In relation to the airflow channels and the airflow directions, several embodiments have been discussed above. Here below, some further aspects and embodiments are discussed.

In embodiments, the direction of the airflow may at least partly be defined by fins and/or (slanted) (end) edges. In specific embodiments, the direction of the airflow may essentially be defined by fins and/or (slanted) (end) edges.

In embodiments, the direction of the airflow may only partly be defined by fins and/or (slanted) (end) edges, or may even substantially not be defined by fins and/or (slanted) (end) edges, but at least a part of the flow channel upstream of the respective second opening(s).

In yet further embodiments, the impact of the airflow channel on the direction of the airflow may essentially be determined by the part of the airflow channel upstream of the respective electrode: the downstream part may have no substantial impact on the direction of the airflow direction.

In specific embodiments, the airflow channel (be it the first airflow channel or the second airflow channel) may have a substantially radial first channel part and a substantial tangential second channel part. For instance, the first part may have a higher curvature than the second part. In other embodiments, the substantially radial first channel part may substantially be straight and the substantial tangential second channel part may substantially be straight. The respective electrode may be at least partly configured in the second channel part, like in embodiments between about the substantially radial first channel part and the substantial tangential second channel part, or just downstream of the substantial tangential second channel part.

In embodiments, the substantially radial first channel part of the airflow channel (be it the first airflow channel or the second airflow channel) may have a first channel part axis which may have a first angle θ1 with a plane perpendicular to the device axis. Further, the substantially tangential part of the airflow channel may have a second channel part axis which may have a second angle θ2 with the plane perpendicular to the device axis. The first channel part axis and the second channel part axis may have a mutual angle θ3. The first angle θ1 and the second angle θ2 may each individually be selected from the range of about 0-85°. Especially, θ1+θ2>0°, such as θ1+θ2≥5°, such as θ1+θ2≥15°, like in embodiments 5°≤θ1+θ2≤85°, such as 10°≤θ1+θ2≤75°. Further, in embodiments, 30°≤θ3≤175°, such as 45°≤θ3≤135°, like about 60°≤θ3≤135°, such as even more especially about 75°≤θ3≤135°.

The first airflow channel may comprise a curved airflow channel. The first airflow channel may comprise bends. The bends may be curved or may be defined by two edge parts defining a mutual angle. The first airflow channel may have a channel axis which may be curved and/or which may have angles (other than 180° or 360°). Especially, the part of the channel axis in the first airflow channel downstream of the first electrode may have an essentially straight channel axis. Likewise, the second airflow channel may comprise a curved airflow channel. The second airflow channel may comprise bends. The bends may be curved or may be defined by two edge parts defining a mutual angle. The second airflow channel may have a channel axis which may be curved and/or which may have angles (other than 180° or 360°). Especially, the part of the channel axis in the second airflow channel downstream of the second electrode may have an essentially straight channel axis.

The device may be functionally coupled to a control system or may comprise a control system. Especially, the device may comprise a control system that may be controlled via (an external) user interface, though other options may also be possible (see also below). In such embodiments, the control system comprised by the device may be a slave control system, which is here further indicated as “control system”.

Especially, the control system may be configured to control one or more of the air ionizer device and the air flow device and the light generating device. For instance, this may be done in dependence of one or more of an input signal of a user interface, a sensor signal, and a timer. Therefore, especially the system may further comprise a control system, wherein the control system may be configured to control the air ionizer device and the air flow device in dependence of one or more of an input signal of a user interface, a sensor signal, and a timer. Alternatively or additionally, the control system may be configured to control the light generating device in dependence of one or more of an input signal of a user interface, a sensor signal, and a timer.

Controlling may imply controlling the potential to the electrodes. Alternatively or additionally, control may also imply controlling a variation in a ratio between positive and negative ions (particles) generated by the electrodes.

Alternatively or additionally, controlling may also imply controlling the respective flows. Alternatively or additionally, control may also imply controlling on-off periods, controlling in dependence of sensor signals, etc. In embodiments, controlling the potential to the electrodes may comprise controlling the voltage to the respective electrodes, controlling a variation in the voltage to the respective electrodes, etc. In embodiments, controlling the respective flows may comprise controlling the respective volumetric ion flow rates.

In specific embodiments, the control system may be configured to select the first operational mode and the second operational mode in dependence of one or more of an input signal of a user interface, a sensor signal, and a timer. For instance, would be sensed that the treatment is not efficient enough, one or more of the potential differences and the flows may be increased. Or, for instance would be sensed that the number of persons in a room increase, also one or more of the potential differences and the flows may be increased. However, would for instance be sensed that over a relative long period there is no presence of people or animals (especially in embodiments pets), one or more of the potential differences and the flows may be decreased, or even switched of. Hence, in specific embodiments the control system may be configured to control the voltage differences relative to the mutual ground to the set of electrodes and the airflows in dependence of one or more of an input signal of a user interface, a sensor signal, and a timer.

Hence, in an operational mode the ionizer device (and optionally also the air flow device) may be operated in a pulsed mode.

This may in embodiments imply that the first electrode may be provided with a pulsed potential (difference), but during a pulse is essentially always configured to generate positively charged particles. Alternatively or additionally, this may in embodiments imply that the second electrode may be provided with a pulsed potential (difference), but during a pulse is essentially always configured to generate negatively charged particles. A positively charged electrode may generate positively charged particles and a negatively charged electrode may generate negatively charged particles.

In yet other embodiments, however, this may imply that the first electrode during a first pulse period is configured to generate positively charged particles and during a second pulse period (earlier or later in time than the first pulse period) is configured to generate negatively charged particles. Alternatively or additionally, this may also in embodiments imply that the second electrode during the first pulse period is configured to generate negatively charged particles and during the second pulse period (earlier or later in time than the first pulse period) is configured to generate positively charged particles. However, other operational modes may also be possible.

Therefore, the first part and second part are not necessarily bound to specific charges. In embodiments, only during an operational mode they may be bound during a specific time to specific charges. Hence, in embodiments the device may be configured to provide during operational modes of the air ionizer (and the airflow device) always positively charged particles via the first part of the second openings and negatively charged particles via the second part of the second openings. However, in other embodiments the electrodes may be operated with a voltage difference that changes in time, which may imply that in embodiments the device may be configured to provide during an operational mode of the air ionizer (and the airflow device) positively charged particles via the first part of the second openings during a first period and optionally negatively charged particles via the second part of the second openings during the same first period and during an operational mode of the air ionizer (and the airflow device) negatively charged particles via the first part of the second openings during a second period and optionally positively charged particles via the second part of the second openings during the same second period.

As indicated above, in embodiments the system may further comprise a control system, wherein the control system may be configured to control one or more of the air ionizer and the air flow device, in embodiments in dependence of one or more of a user interface, a sensor, and a timer. Especially, the air ionizer and the air flow device may be controlled individually. Especially, the timer may comprise in embodiments a time scheme. In embodiments, the control system may control one or more of the air ionizer and the air flow device, in embodiments in dependence of a clock module (or timer). The clock module may provide a time scheme. The control system, especially in combination with the sensor, may provide the system with instructions for changing from a first operational mode to a second operational mode, depending on signals from the sensor. In embodiments, the control system may receive signals from a user interface, such that a user can control one or more of (i) potential on the first electrode, (ii) potential on the second electrode, and (iii) first volumetric flow rate, and (iv) second volumetric flow rate.

In embodiments, the sensor may comprise one or more sensors selected from the group comprising: a movement sensor, a presence sensor, a distance sensor, an ion sensor, a gas sensor, a virus sensor, an airflow sensor, a radiation sensor, a bacterium sensor, and a communication receiver.

In embodiments, the control system may be configured to control the volumetric ion flow rate Q in dependence of the sensor. Especially, the control system may in embodiments increase the volumetric ion flow rate Q when movement or presence is detected. Vice versa may also be the case: in embodiments, the volumetric ion flow rate Q may be decreased when no movement or presence is detected. In alternative embodiments, the control system may in embodiments decrease the volumetric ion flow rate Q when movement or presence is detected. Thus, in specific embodiments, the sensor may comprise one or more of a presence sensor and a movement sensor, and wherein the control system may be configured to control the volumetric ion flow rate Q in dependence of the sensor.

The term “sensor” may also refer to a plurality of (different) sensors.

In embodiments, the ion concentration may be defined as the number of ions per cubic centimeter. The ion concentration may be quantified using an air ion counter.

Alternatively or additionally, controlling may also imply controlling one or more spectral properties of the device light, like spectral power distribution, color point, correlated color temperature, color rendering index, etc.

In embodiments, the device may in an operational mode be configured to generate white device light. In other embodiments, in (another) operational mode, the device may be configured to generate colored light.

The terms “light” and “radiation” are herein interchangeably used, unless clear from the context that the term “light” only refers to visible light. The terms “light” and “radiation” may thus refer to UV radiation, visible light, and IR radiation. In specific embodiments, especially for lighting applications, the terms “light” and “radiation” refer to (at least) visible light.

The term “controlling” and similar terms especially refer at least to determining the behavior or supervising the running of an element. Hence, herein “controlling” and similar terms may e.g. refer to imposing behavior to the element (determining the behavior or supervising the running of an element), etc., such as e.g. measuring, displaying, actuating, opening, shifting, changing temperature, etc. Beyond that, the term “controlling” and similar terms may additionally include monitoring. Hence, the term “controlling” and similar terms may include imposing behavior on an element and also imposing behavior on an element and monitoring the element. The controlling of the element can be done with a control system, which may also be indicated as “controller”. The control system and the element may thus at least temporarily, or permanently, functionally be coupled. The element may comprise the control system. In embodiments, the control system and element may not be physically coupled. Control can be done via wired and/or wireless control. The term “control system” may also refer to a plurality of different control systems, which especially are functionally coupled, and of which e.g. one control system may be a master control system and one or more others may be slave control systems. A control system may comprise or may be functionally coupled to a user interface.

The control system may also be configured to receive and execute instructions form a remote control. In embodiments, the control system may be controlled via an App on a device, such as a portable device, like a Smartphone or I-phone, a tablet, etc. The device is thus not necessarily coupled to the lighting system, but may be (temporarily) functionally coupled to the lighting system.

Hence, in embodiments the control system may (also) be configured to be controlled by an App on a remote device. In such embodiments the control system of the lighting system may be a slave control system or control in a slave mode. For instance, the lighting system may be identifiable with a code, especially a unique code for the respective lighting system. The control system of the lighting system may be configured to be controlled by an external control system which has access to the lighting system on the basis of knowledge (input by a user interface of with an optical sensor (e.g. QR code reader) of the (unique) code. The lighting system may also comprise means for communicating with other systems or devices, such as on the basis of Bluetooth, WIFI, LiFi, ZigBee, BLE or WiMAX, or another wireless technology.

Hence, in embodiments, the control system may control in dependence of one or more of an input signal of a user interface, a sensor signal (of a sensor), and a timer. The term “timer” may refer to a clock and/or a predetermined time scheme.

Especially, in embodiments the device may be a retrofit lamp.

The device may be part of or may be applied in e.g. office lighting systems, household application systems, shop lighting systems, home lighting systems, accent lighting systems, spot lighting systems, theater lighting systems, decorative lighting systems, portable systems, automotive applications, etc.

For the device it may be advantageous to be located at a ceiling or otherwise over a floor, such as suspended from a roof, etc. In this way, the amount of obstruction by other elements, like chairs, desks, cupboards, cubicle walls, etc., may be minimized and large areas may be treated (with one or more units). As many locations are provided with downlighting based systems, these might be an efficient way to incorporate the herein proposed device. Thus, in embodiments, the device may be incorporated in a downlight based system (“downlight”), like a (downlighting) luminaire or (downlighting) spotlight.

The device may be used to treat air in a space. The term “space” may for instance relate to a (part of) hospitality area, such as a restaurant, a hotel, a clinic, or a hospital, etc. The term “space” may also relate to (a part of) an office, a department store, a warehouse, a cinema, a church, a theatre, a library, etc. However, the term “space” may also relate to (a part of) a working space in a vehicle, such as a cabin of a truck, a cabin of an air plane, a cabin of a vessel (ship), a cabin of a car, a cabin of a crane, a cabin of an engineering vehicle like a tractor, etc. The term “space” may also relate to (a part of) a working space, such as an office, a (production) plant, a power plant (like a nuclear power plant, a gas power plant, a coal power plant, etc.), etc. For instance, the term “space” may also relate to a control room, a security room, etc. In yet other embodiments, the term “space” may also relate to a toilet room or bathroom. In yet other embodiments, the term “space” may also relate to an elevator. In embodiments, the term “space” may also refer to a conference room, a school room, an indoor hallway, an indoor corridor, an indoor space in an elderly home, an indoor space in a nursing home, etc. In embodiments, the term “space” may refer to an indoor sport space, like a gym, a gymnastics hall, in indoor ball sport space, a ballet room, a swimming pool, a changing room, etc. In embodiments, the term “space” may refer to an (indoor) bar, an (indoor) disco, etc.

Especially for indoor areas that are larger than the reach of a single device, such as offices, public transport, cinema's, restaurants, shops, etc., multiple device devices may be applied.

Hence, in an aspect also a system comprising a plurality of the herein described devices is provided. Especially, such system may comprise a (system) control system, configured to control the plurality of devices.

Hence, in embodiments the invention also provides a light generating system comprising the device as defined herein, wherein the light generating system comprises a control system configured to control the device in dependence of one or more of an input signal of a user interface, a sensor signal, and a timer.

Hence, in embodiments, the system may comprise a grid of a devices. Such grid may be installed in a roof or ceiling. In embodiments, the individual device may be functionally connected to the control system. In embodiments, the individual device in the grid may comprise a sensor, especially one or more of a radiation sensor and an air flow sensor. In embodiments, a first device may adjust its settings based on sensor signals. In embodiments, the individual devices, especially the control systems thereof, may communicate with one another. The individual devices may comprise means for communicating with other units, systems or devices, such as on the basis of Bluetooth, WIFI, LiFi, ZigBee, BLE or WiMAX, or another wireless technology. In specific embodiments, settings of a first device of the grid may depend on the settings of a second device of the grid.

In embodiments, the device radiation may be directed downwards, especially to a floor.

In embodiments, the grid of devices may be configured such that a first airflow of a first device may be directed away from a second airflow of a second device. Likewise, the grid of devices may be configured such that a second airflow of a first device may be directed away from a first airflow of a second device. In this way, the plurality of devices may enhance one another instead of neutralizing one another.

In an aspect, the invention further provides a method for treating at least part of a space, wherein the method comprises providing one or more of the first airflow and a second airflow; especially both, in the space. The first airflow may comprise positively charged particles or negatively charged particles, and the second airflow may comprise negatively charged particles or positively charged particles. In another aspect, the invention comprises a method for treating air.

In this way, the method may provide one or more of disinfection of pathogens, removal of particles and dust, and removal of odors. Especially, the treatment of the air may comprise disinfection of (the) air.

Therefore, in yet a further aspect the invention provides a method for one or more of (i) treating a gas and (ii) providing light, wherein the method comprises operating the device as defined herein or the light generating system as defined herein.

The embodiments described above in relation to the system of the present invention, may also apply for the method of the invention.

In embodiments, the device may provide several operational modes. In embodiments, in a first operational mode the device may provide device light. In embodiments, in a second operational mode, the device may provide an airflow with positively charged particles. In embodiments, in a third operational mode, the device may provide an airflow with negatively charged particles. In embodiments, the first operational mode, the second operational mode and the third operational mode do not overlap in time. In other embodiments, the first operational mode may at least partly overlap in time with one or more of the second operational mode and the third operational mode. In vet other embodiments, the second operational mode may at least partly overlap in time with the first operational mode. In yet further embodiments, the third operational mode may at least partly overlap in time with the first operational mode. In embodiments, the second operational mode and the third operational mode overlap in time. Hence, in embodiments the second operational mode and the third operational mode may only be executed simultaneously. In yet other embodiment, the second operational mode and the third operational mode partly overlap in time. In yet other embodiment, the second operational mode and the third operational mode do not overlap in time. In specific embodiments, the second operational mode and the third operational mode may (only) be executed sequentially. Hence, in specific embodiments the device may be operated in that alternating airflows are provided, wherein airflows with positively charged particles alternate with airflows with negatively charged particles. In embodiments, the second operational mode and/or the third operational mode (especially both) may only be executed in dependence of a sensor signal of a sensor. In other embodiments, the second operational mode and/or the third operational mode (especially both) may only be executed in dependence of a user instruction via a user interface. In specific embodiments, the second operational mode and/or the third operational mode (especially both) may be executed in dependence of one or more of a user instruction via a user interface and a sensor signal of a sensor. However, in specific embodiments, the second operational mode and/or the third operational mode (especially both) may be executed on the basis of a timer. The term “operational mode” may in embodiments also refer to a plurality of operational modes. For instance, the first operation mode may in embodiments exclusively refer to the generation of white device light, whereas in other embodiments it may refer to operational modes of a device which allows color control of the device light, thereby allowing different (first) operational modes with different spectral power distributions of the device light.

Hence, the term “light emitting area” does not imply that during operation of the device always the light generating device is operated an generated light that may escape via the light emitting area (in the first operational mode).

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1 schematically depicts an embodiment of the device:

FIGS. 2a-2j schematically depict some aspects and embodiments:

FIG. 3 schematically depict yet some further aspects and embodiments:

FIG. 4 schematically depicts an application; and

FIG. 5 schematically depicts some possible pulse schemes.

The schematic drawings are not necessarily to scale.

### DETAILED DESCRIPTION OF THE EMBODIMENTS

FIG. 1 schematically depicts an embodiment of a device 1200. The device 1200 comprises a light generating device 100, an air ionizer device 500, and an airflow device 600.

Further, the device 1200 comprises one or more first openings 710 and one or more second openings 720. Especially, the device 1200 comprises a front part 750 comprising the one or more second openings 720. The air ionizer device 500 may be configured to generate in an operational mode positively charged particles at a first electrode 510 and negatively charged particles at a second electrode 520. Further, the airflow device 600 may be configured to generate in the operational mode a first airflow 610 entraining the positively charged particles and a second airflow 620 entraining the negatively charged particles. Reference d indicates a mutual electrode distance, between the first electrode 510 and the second electrode 520 (not taking into account physical barriers).

Especially, the one or more first openings 710 may be configured upstream of the airflow device 600 and the electrodes 510,520. Further, in embodiments a first part 721 of the one or more second openings 720 may be configured downstream of the airflow device 600 and the first electrode 510. Yet further, in embodiments a second part 722 of the one or more second openings 520 may be configured downstream of the airflow device 600 and the second electrode 520. As schematically depicted, the first part 721 and the second part 722 may be spatially separated.

Further, the device 1200 may comprise a light emitting area 150, from which during operation of the light generating device 100 device light 101 escapes from the device 1200. Especially, the front part 750 comprises the light emitting area 150.

In embodiments, the device 1200 may comprise one or more light sources, especially one or more solid state light sources 10. Hence, the light generating device 100 may comprise one or more (solid state) light sources 10.

The device 1200 may comprise an enclosure arrangement 700. The enclosure arrangement 700 may at least partly enclose the light generating device 100, the air ionizer device 500, and the airflow device 600. Further, in embodiments the device 1200 may comprise a first end 1210 and a second end 1220.

Especially, the light emitting area 150 may be configured closer to the first end 1210 than to the second end 1220. Further, in embodiments the enclosure arrangement 700 may comprise the one or more first openings 710 configured between the first end 1210 and the second end 1220.

The device 1200 may comprise electrical connectors 1205 and electronics 1250, wherein the electronics 1250 are at least partly enclosed by the enclosure arrangement 700. In embodiments, the electronics 1250 may be functionally coupled to the light generating device 100, the air ionizer device 500, the airflow device 600, and the electrical connectors 1205. Especially, the electrical connectors 1205 may be configured closer to the second end 1220 than the first end 1210. The term “electronics” 1250 may also refer to a plurality of electronics which may be configured at different locations within the enclosure arrangement 700 (but which may be functionally coupled to each other in embodiments).

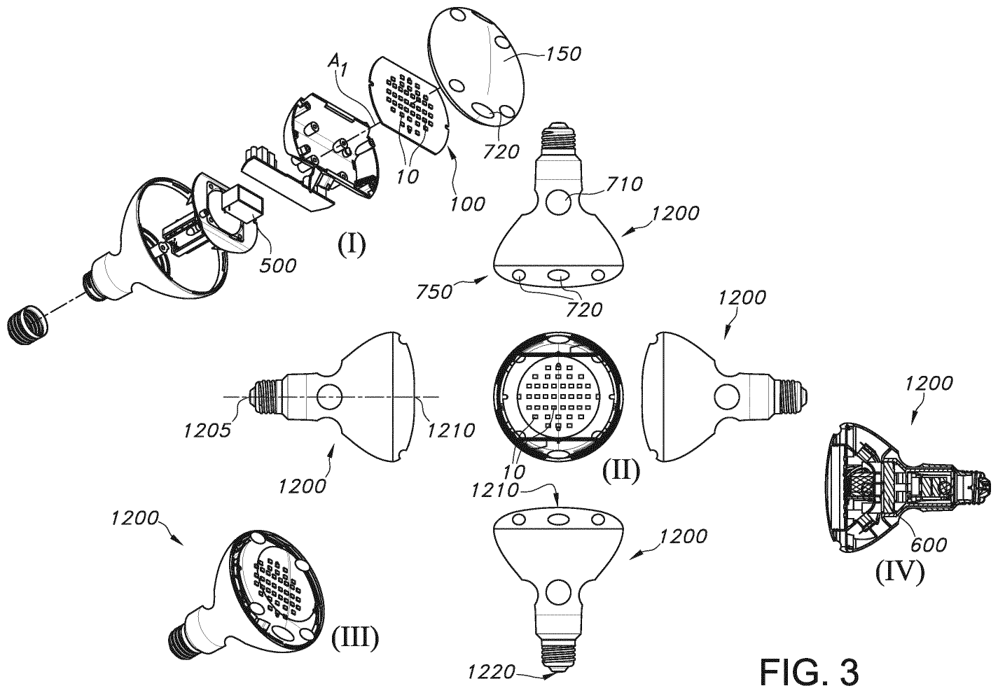

In specific embodiments, see also FIG. 3, the device 1200 may be a retrofit lamp.

In specific embodiments, the device 1200 may in an operational mode be configured to generate white device light 1201. However, alternatively or additionally in an (other) operational mode, the device 1200 may be configured to generate colored light 1201.

Further, the device may comprise a front element 760. Especially, in embodiments the front element 760 may comprises an optical element 770, especially comprising the light emitting area 150. The optical element 770 may be configured downstream of the one or more (solid state) light sources 10. In embodiments, the first part 721 and the second part 722 may at least partly enclose the optical element 770.

In embodiments, the optical element 770 may comprise a diffuser.

The front part 750) may have an essentially circular cross-section (which may especially be defined perpendicular to the device axis) over its entire length. Further, especially in embodiments the middle part may have essentially circular cross-section (which may especially be defined perpendicular to the device axis) over its entire length. Yet further, especially in embodiments the end part may have essentially circular cross-section (which may especially be defined perpendicular to the device axis) over its entire length.

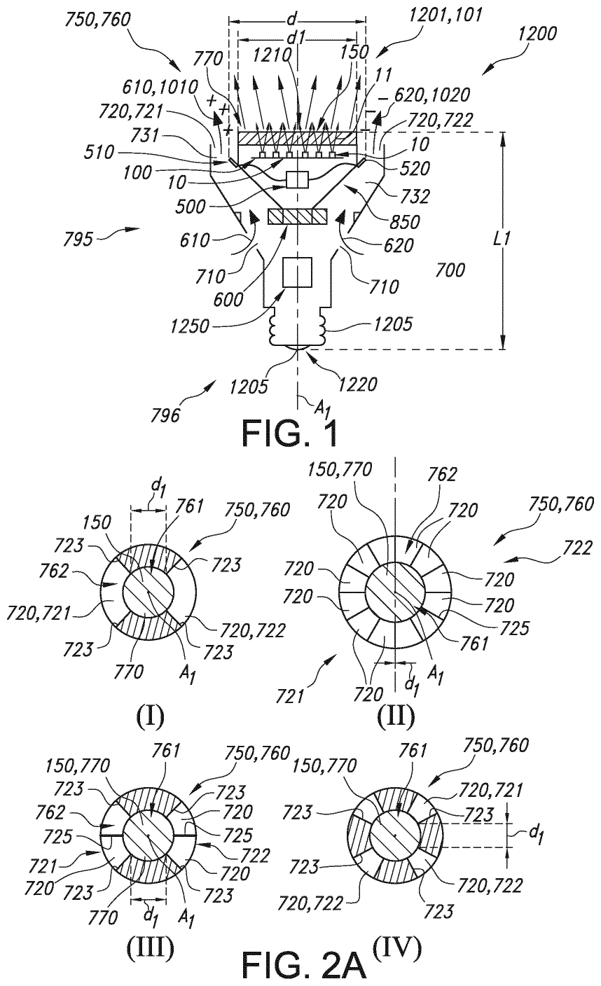

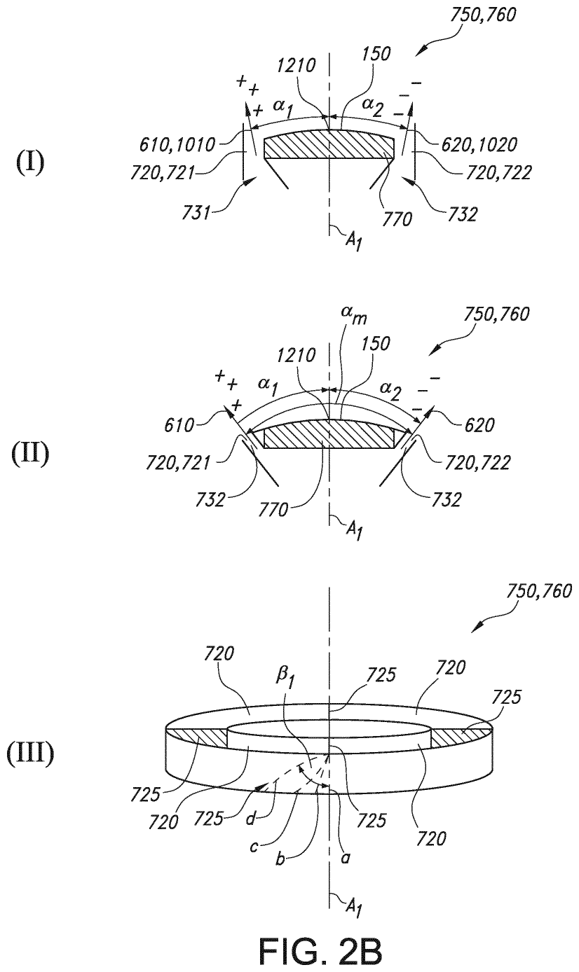

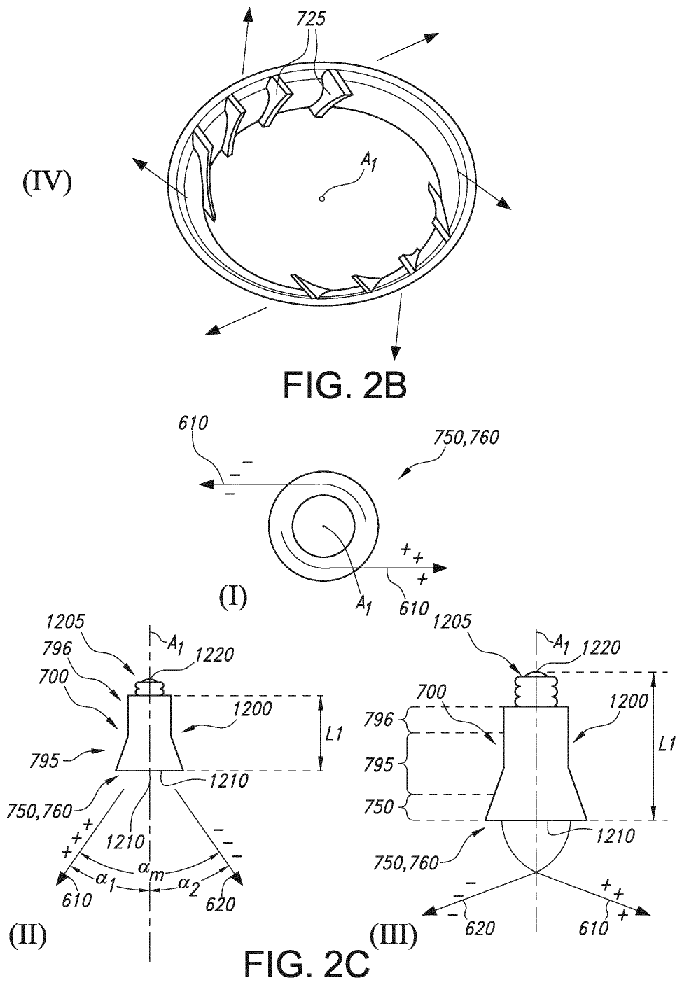

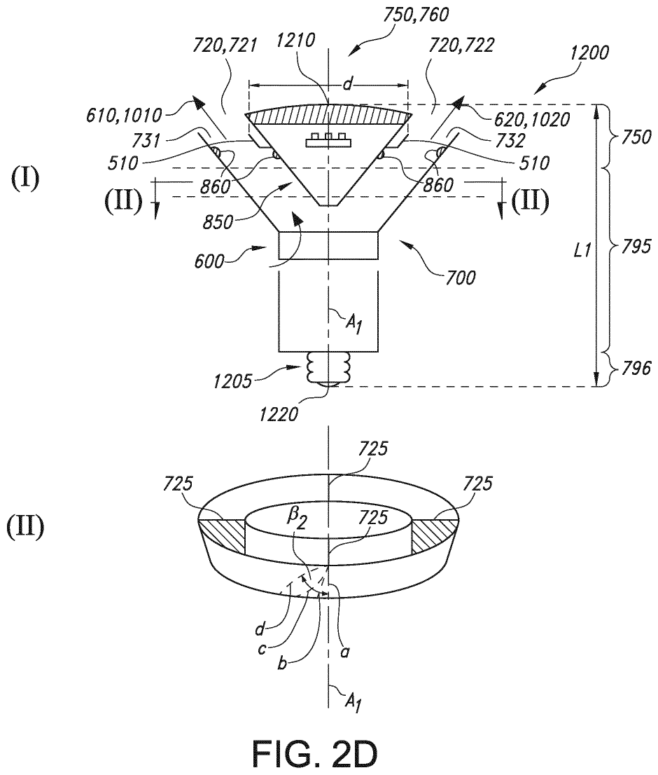

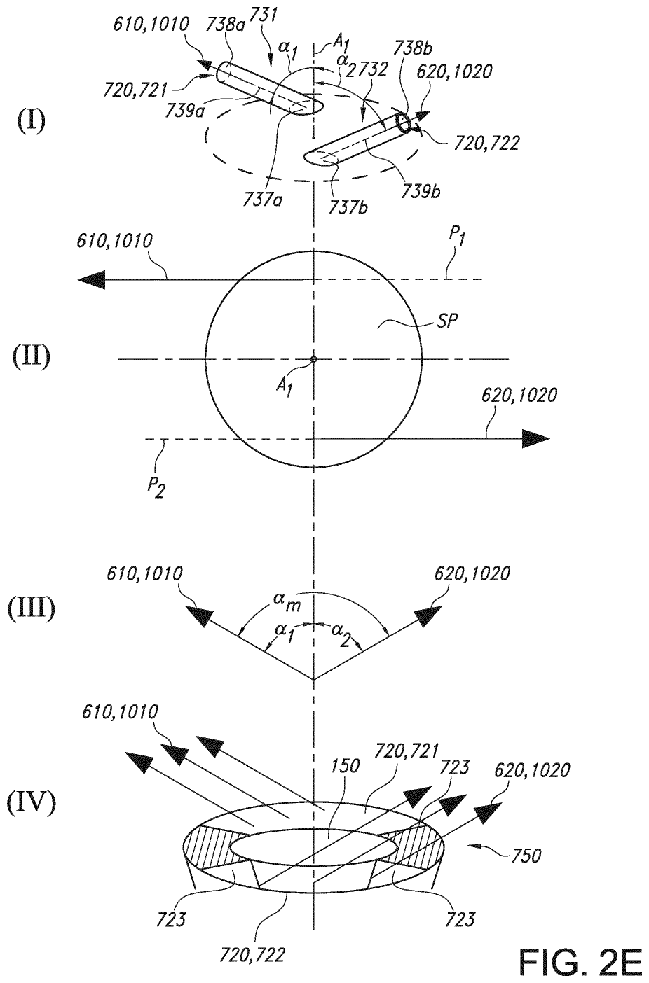

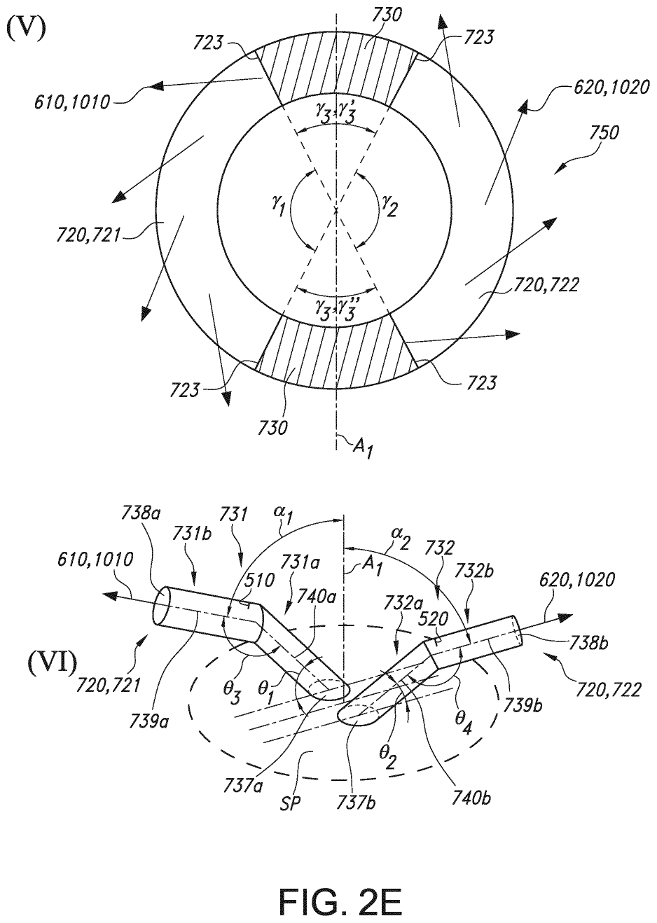

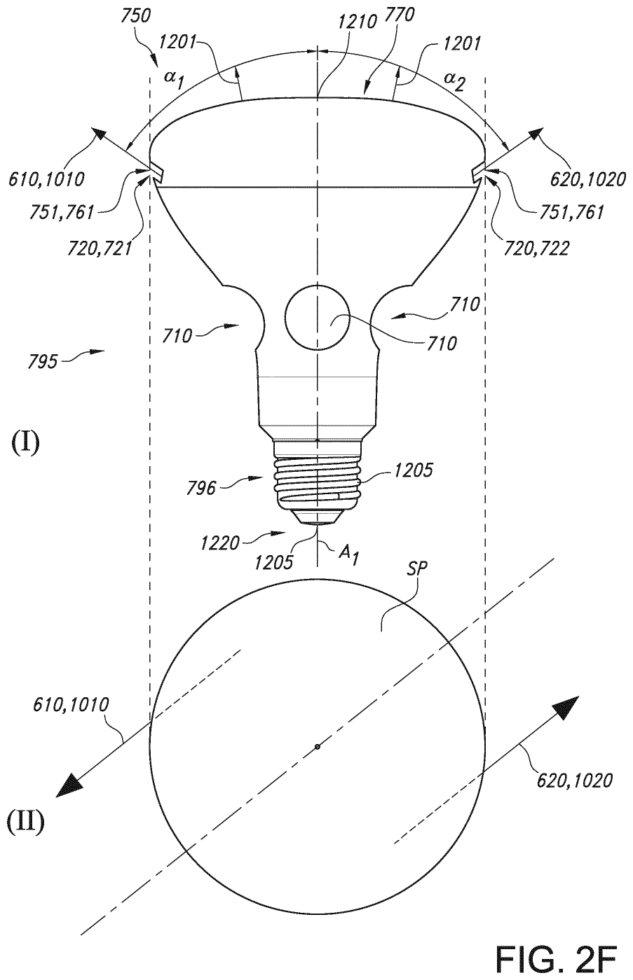

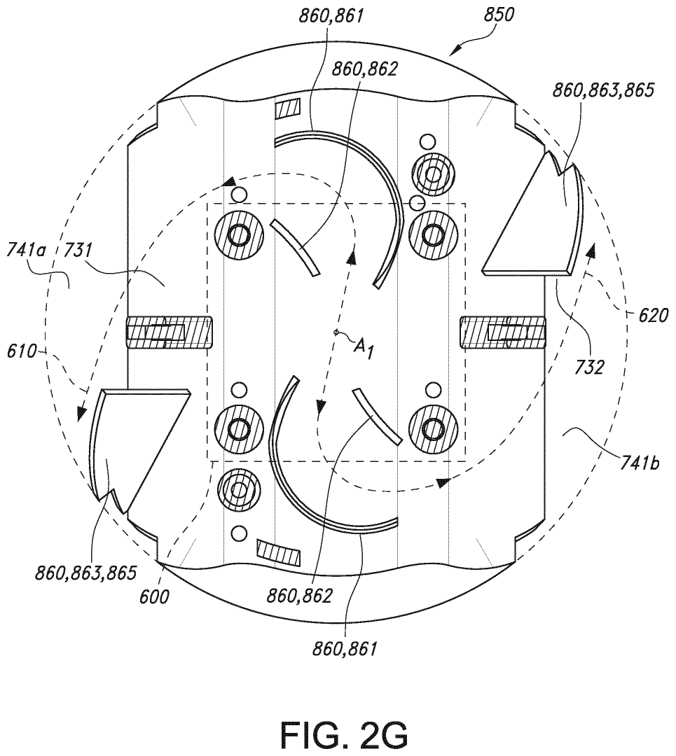

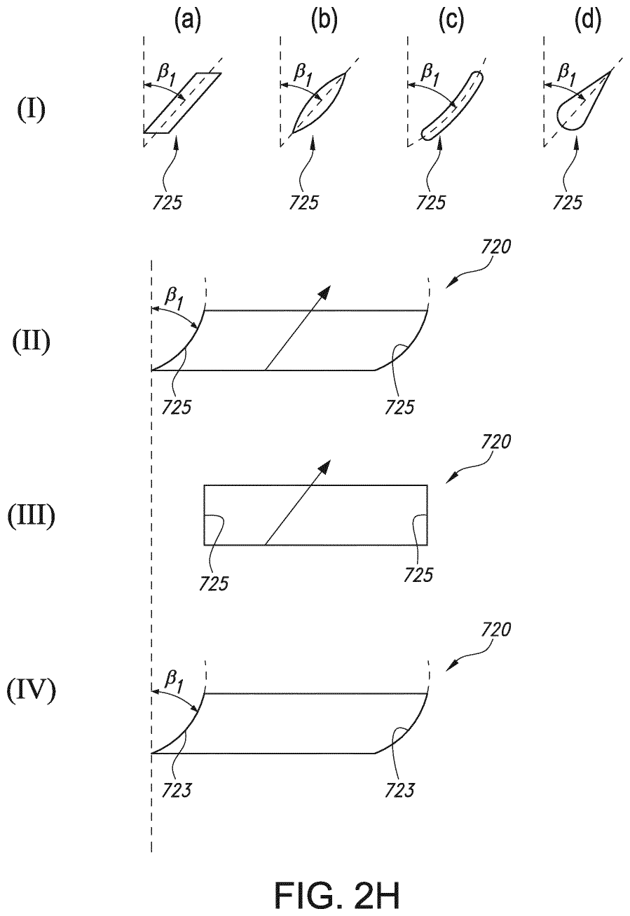

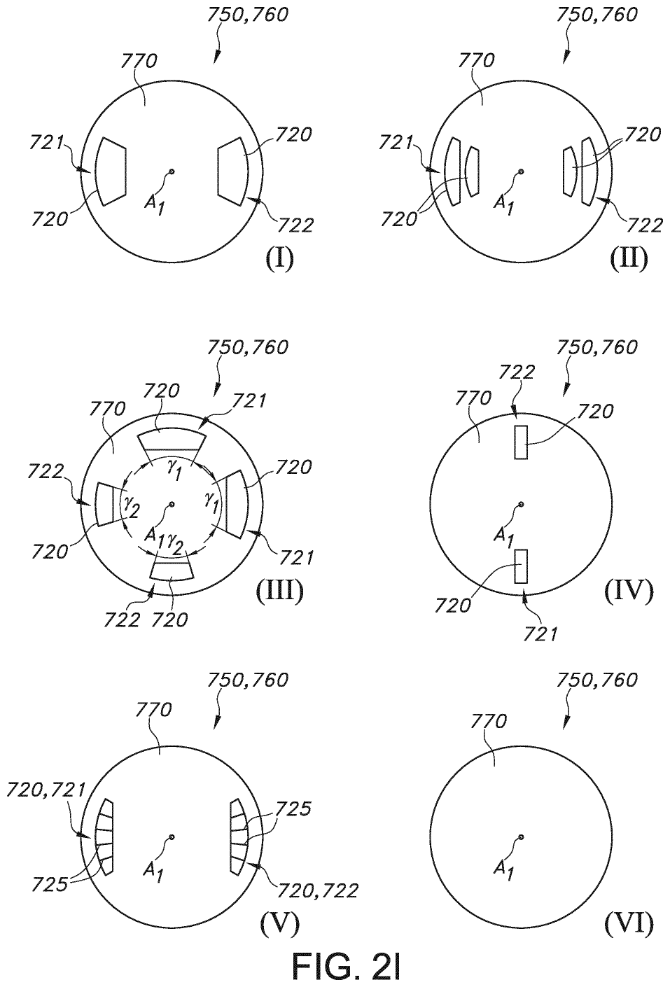

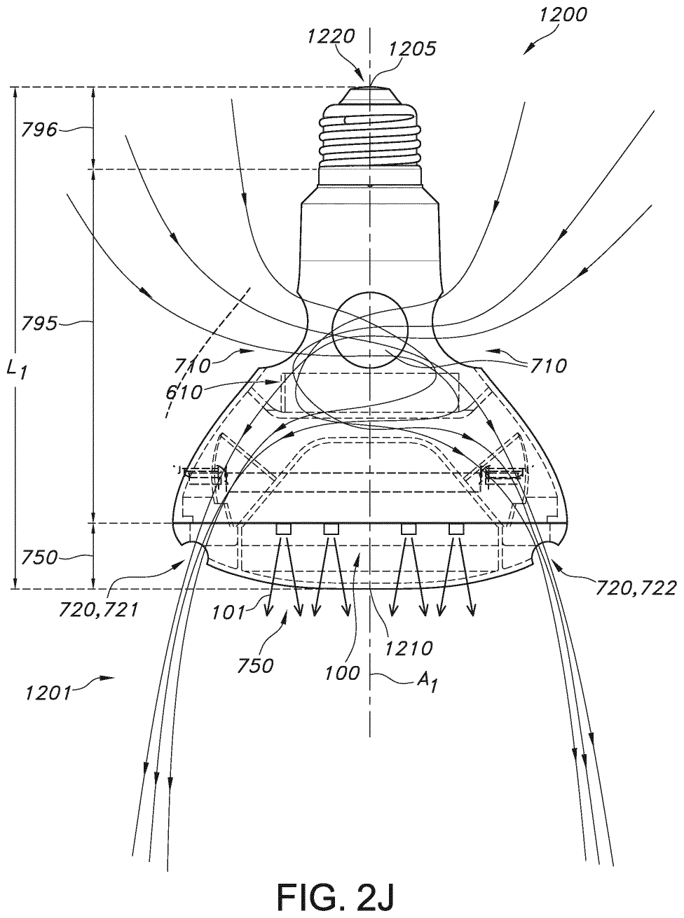

Referring to FIG. 1, but e.g. also to FIG. 2a, in embodiments, the optical element 770 may at least partly be surrounded by the first part 721 and the second part 722 (of the second openings 720). In specific embodiments, the optical element 770 may completely be surrounded by the first part 721 and the second part 722.

In specific embodiments, the first part 721 may comprise 1-4 second openings 720), and the second part 722 may comprises 1-4 second openings 720.

In embodiments, the front element 760 may comprise a central part 761 and a peripheral part 762. Especially, in embodiments the central part 761 may comprise the optical element 770, and the peripheral part 762 may comprise the first part 721 and the second part 722. In embodiments, the front element 760 may be a monolithic element. However, this is not necessarily the case. In embodiments, the front element 750 may be molded, but other embodiments may also possible. The front element 750 may also be made of several parts, e.g. central part and ring, or other configurations (see also below).

Reference L1 indicates the total length along the device axis A1, between the first end 1210 and the second end 1220. Reference d1 indicates a shortest distance between the first part and the second part, taking into account physical barriers. The shortest distance d1 may be about 5-150 mm, such as selected from the range of 5-80 mm, like at least 10 mm. In specific embodiments, the shortest distance d1 between the first part and the second part, taking into account physical barriers, may at least be about 15 mm.

As shown, there is tapering part between the first end 1210 and the second end 1220, which may e.g. be comprised by the middle part 795.

The light sources 10 are especially configured to generate light source light, which is indicated with reference 11. Especially, the light sources are solid state light sources. This light source light 11 may be used as such or may at least partly be converted by a luminescent material into luminescent material light (not indicated). Hence, light 101 from the light generating device 100 may comprise one or more of light source light 11 and converted light source light (luminescent material light). The light from the light generating device, i.e. light generating device light is indicated with reference 101. Light escaping from the light generating device 1200 may be indicated as device light 1201, and may essentially consist of the light generating device light 101, i.e. may comprise at least part of the light source light 11 and/or at least part of the luminescent material light (should a luminescent material be applied). Note that not necessarily a luminescent material may be applied. Further, solid state light sources may also comprise PC LEDs, where the light source light 11 may already comprise luminescent material light.

Reference 850 refers to a support element, which is discussed also below.

The air flow device 600, such as a fan, may refer to a single airflow device, or to a plurality of airflow devices, like a plurality of fans.

Two channels are provided downstream of the airflow device 600, a first airflow channel 731 and a second airflow channel 732. The first electrode 510 may at least partly be configured in the first airflow channel 731 and the second electrode 520 may at least partly be configured in the second airflow channel 732.

Reference 750 refers to front part, reference 795 refers to a middle part, and reference 796 refers to an end part. The front part 750 may comprise the first end 1210. The end part 796 may comprise the second end 1220. The end part 796 may e.g. comprise a screw cap (herein depicted) or other type of cap (such as a bayonet base). In embodiments, the front part 750, the middle part 795, and the end part 796 may essentially enclose all functional components of the device 1200.

Here, the front part 750 is schematically depicted with a flat part comprising the light emitting area 150. However, the front part may also have a curved light emitting area 150, such as a curved front element 760 (see also below).

Referring to FIG. 2a, four non-limiting embodiments of a front part 750, or at least part thereof, are schematically depicted (see also above). As indicated above, the front part 750 may comprise the front element 760.

Embodiment I of FIG. 2a schematically depicts an embodiment wherein the two parts 721,722 each comprise a single second opening 720. Further, the second openings 720 do not fully surround the light emitting area 150. The two parts 721,722 are configured opposite to each other, relative to the device axis A1.

Effectively, the schematically depicted circle may be divided in a first circle section comprising the first part of the one or more second openings and a second circle section comprising the second part of the one or more second openings, and a third circle section not comprising second openings. Here, the third circle section comprise two smaller circle section. The angular part defined by the first circle section may be about 90°, the angular part defined by the second circle section may be about 90°, and the angular part defined by the third circle section may be about 2*90°.

Embodiment II of FIG. 2a schematically depicts an embodiment wherein the second openings 720 essentially fully surround the light emitting area 150. One or more of the second openings 720, or in specific embodiments all, may be comprised by the two parts 721,722. Note that the upstream airflow channels may be configured such, that most of the respective airflow leaves the device only via a limited number of second openings 720. FIG. 2a, embodiment II schematically depicts an embodiments wherein there may be a ring of (one or more, especially) a plurality of second openings. In such embodiments, there may be no third circle section (see also below).

Embodiment III of FIG. 2a schematically depicts an embodiment wherein the two parts 721,722 each comprise a two second openings 720. The two second openings 720 are separated by a fin 725. Further, the second openings 720 do not fully surround the light emitting area 150. The two parts 721,722 are configured opposite to each other, relative to the device axis A1. Hence, the first airflow may leave the device via the two second openings 720 at the left right in the schematic drawing of embodiment III, and the second airflow may leave the device via the two second openings 720 at the right in the schematic drawing of embodiment III. The angular part defined by the first circle section may be about 90°, the angular part defined by the second circle section may be about 90°, and the angular part defined by the third circle section may be about 2*90°.

Embodiment IV of FIG. 2a schematically depicts an embodiment comprising two first parts 721 and two second parts 722, each comprise a single second opening 720. Further, the second openings 720 do not fully surround the light emitting area 150.

The angular part defined by the first circle section may be about 2*35°, the angular part defined by the second circle section may be about 2*35°, and the angular part defined by the third circle section may be about 4*55°.

In embodiments, the two first part 721 and the two second part 722 may together provide a first part 721 and a second part 722, respectively. In such embodiments, e.g. the first airflow and the first electrode(s) related to the first parts may be controlled as set, though this is not necessarily the case. Likewise, in such embodiments, e.g. the second airflow and the second electrode(s) related to the second parts may be controlled as set, though this is not necessarily the case. However, it may also be possible that the two first parts are functionally coupled to different first electrodes, that may individually controlled. Likewise, it may also be possible that the two second parts are functionally coupled to different second electrodes, that may individually controlled. It may also be the case that two first flow channels are functionally coupled to (first) different airflow devices, which may be controlled individually, and/or that two second flow channels are functionally coupled to different (second) airflow devices which may be controlled individually. In the schematically depicted embodiments, clockwise the configuration may ++−−. However, in alternative embodiments, clockwise the configuration may be +−+−.

Note that indicates like “++−−” may (also) be used to indicate the configuration of the airflow channels, like two adjacent airflow channels that may at the same time be used to provide positively charged particles and two adjacent airflow channels that may at the same time be used to provide negatively charged particles. However, this may not exclude that in other operational modes, one or more of the ++−− charges (for the same channel(s)) may be opposite, like e.g. −+ or +−+−, etc.

Referring to embodiments I and III of FIG. 2a, the shortest distance (d1) between those second parts, taking into account physical barriers, may be about 5-150 mm, such as selected from the range of 5-80 mm, like at least 10 mm.

In embodiments II and IV the value of the shortest distance may depend on which second opening is used as outlet for the airflow. Further, it may even be possible that different airflows, i.e. airflows that differ in the charged particles that are entrained, may be provided at different times. When the difference in time is large enough between the different airflows, annihilation of the charge by particles of one airflow by particles of another airflow may be prevented. Therefore, especially during an operational mode wherein at the same time (or optionally within about 5 seconds of each other) the first airflow and the second airflow are provided from different second parts, the shortest distance d1 between those second parts, taking into account physical barriers, may be about 5-150 mm, such as selected from the range of 5-80 mm, like at least 10 mm.

Referring to embodiments I-IV of FIG. 2a, the first part of the second openings and the second part of the second openings, may in embodiments may surround the device axis over 360°. In other embodiments, however, the first and second part of the one or more second openings may surround the device axis over less than 360°, such as selected from the range of 10-270°, like in embodiments selected from the range of 20-180°.

Referring also to FIG. 2b, the device 1200 may comprise a device axis A1. In embodiments, the first airflow 610 may have a first direction 1010 having a first angle α1 relative to the device axis A1, and the second airflow 620 may have a second direction 1020 having a second angle α2 relative to the device axis A1. In embodiments, the first angle α1 and the second angle α2 may independently be selected from the range of 0-90°. However, in specific embodiments one or more of the first angle α1 and the second angle α2 may also be larger than 90°.

Especially, in embodiments the first direction 1010 and the second direction 1020 have a mutual angle αm selected from the range of 5°-180°. However, other mutual angles may also be possible. Especially, the mutual angles are not smaller than bout 4° and not larger than about 356°.

Especially, the direction of the airflow can especially be defined as the direction where the respective airflow (escaping from a first part of the one or more second openings or from the second part of the one or more second opening) is maximal.

Embodiments I and II are cross-sectional views of at least part of the front part 150 of the device 1200. Here, by way of example the optical element 770 may provide a curved light emitting area 150.

Embodiment III of FIG. 2b schematically depicts an embodiment with four openings 720, separated by four fins 725. Schematically, it is shown that the fins 725 may be configured under angles, indicated with reference β1. Four examples are showed with dashed lines, with a having an angle β1 with the device axis A1 of 0°, and examples b, c, and d having angles β1 between smaller than 90° but larger than 0°. Reference b refers to a straight fin 725 and references c and refer to curved fins 725, which may be convex or concave. These option may also apply to the other fins 725 schematically depicted in embodiment III of FIG. 2b, but for the sake of understanding the drawing, this has not implemented in this drawing. However, an embodiment thereof is schematically depicted in embodiment IV of FIG. 2B. Specific (other) shapes of fins are described elsewhere.

Embodiment IV of FIG. 2B schematically depict four fins 725 at two opposite sides of the device axis A1. The airflow may escape along these fins, and may thus escape from the front part 750 within distinct radial parts, not fully enclosing the device axis A1, like e.g. in embodiments I and III (and in fact also embodiment IV) of FIG. 2a. Here, the fins may have slightly curved faces, which may lead to different angles with the device axis A1 over the curved faces.

Referring to FIG. 2b, embodiments III and IV, one or more of the following may apply: (a) the first part 721 comprises at least one second opening 720 at least partly defined by fins 725, which are not in a same plane as the device axis A1 and which are non-perpendicular to a plane perpendicular to the device axis A1, and (b) the second part 722 comprises at least one second opening 720 at least partly defined by fins 725, which are not in a same plane as the device axis A1 and which are non-perpendicular to a plane perpendicular to the device axis A1.

FIG. 2c schematically depicts in embodiment I how from part of the front part the airflows 610 and 620 may escape (see e.g. also FIG. 2j). The directions may be different (here essentially opposite) and the directions may not be in the same (straight) plane. FIG. 2c, embodiment I, may schematically depict a kind of top view.

Embodiments II and III schematically depict possible side views from different sides. As schematically shown, the airflows 610,620 may have a mutual angle αm, which may e.g. be selected from the range of 5-165°, though in other embodiments may have other values.

FIG. 2d schematically depicts in embodiment I that the airflow channels 731,732 may essentially be without curves, bends, angles downstream of the respective electrodes 510,520. Downstream of the airflow device 600, two channels 731,732 provide the different airflows 610,620 to the respective first part 721 of the one or more second openings 720 and the respective second part 722 of the one or more second openings. In order to create e.g. two airflows which have directions 1010 that are not in the same plane, not only fins may be used at the second openings (see above), but also upstream of the electrode elements may be used to create the desired airflow directions.

Embodiment II of FIG. 2d schematically depicts an upstream part, upstream of the electrodes 510,520, but downstream of the airflow device 600, which upstream part may include also include e.g. fin-like elements or other flow influencing elements.

Referring to FIG. 2e, but in embodiments also to FIGS. 2b, 2c, and 2d, in embodiments the first direction 1010 and the second direction 1020 may not configured in a single plane.

FIG. 2e, embodiment I is a schematically picture, which is not necessarily reflecting a possible embodiment. This schematical drawings is used to show that the airflow directions 1010,1020, which may thus not be in the same plane, may by way of example be produced by two different outlets of tubes which both may not coincide, or at least their tube axes, with the device axis A1. A further variant thereon is schematically depicted in FIG. 2e embodiment VI. The (virtual) tubes generating the air flows, i.e. the (virtual) tubes reflecting the airflow channels 731,732 have inlets 737a,737b, respectively. The (virtual) tubes generating the air flows, i.e. the (virtual) tubes reflecting the airflow channels 731,732 have outlets or exhausts 738a,738b, respectively. The channel axes (or tube axes) are indicated with references 739a,739b, respectively. Hence, as these channel axes 739a,739b may thus not be in the same plane, and may not coincide with the device axis A1.

Note that the (virtual) tubes are used to describe an effective airflow channel, which may be defined by especially the second openings, vanes, opening edges, etc.

A top view of the airflow direction is schematically depicted in embodiment II of FIG. 2e. The mutual angle αₘ may be defined by the respective angles α1 and α2 with the device axis, see embodiment II, FIG. 2e. Reference SP schematically indicates a straight (virtual) plane. References P1 and P2 are virtual elongations of the directions 1010,1020 of airflows 610,620, respectively.

Embodiment III of FIG. 2e schematically depicts the respective angles α1 and α2 with the axis A1, as well as the mutual angle αₘ.

A perspective embodiment of (at least part of) the front part showing two airflows 610,620, having different directions 1010,1020, which are not in the same (straight) plane, is schematically depicted in embodiment IV of FIG. 2e. Hence, whereas FIG. 2d embodiment II may schematically depict an embodiment of at least part of a front part, FIG. 2e embodiment IV may schematically depict an embodiment of at least part of the device closer to the airflow device, e.g. just downstream thereof. Hence, a schematical/abstract depiction of embodiment IV of FIG. 2e may thus be in fact embodiment I of FIG. 2e. Hence, the internal structure of the device and the second openings may in embodiments be selected such as if two tubes are used to generate the airflows, which are configured such that the escaping airflows 610,620 have direction 1010,1020, respectively, which may not coincided with the device axis A1. Thence, such (virtual) tubes (or ducts or airflow channels) may (virtually) be configured offset from the device axis A1 (and having tube axes not in the same plane).

Embodiment V of FIG. 2e schematically depict that in embodiments different radial parts may comprise second openings 720 and other radial parts may not comprise second opening 720. Here, the circular sector that may comprise second openings 720 is defined by respective angles γ1 and γ2: the circular sector not comprising second openings is defined by γ₃=360°−γ1−γ2. Note that the term “circular sector” may also refer to different circular sectors. The third circular sector is indicated with reference 730. Actually, here two second third circular sectors are indicated, each having a respective angle of about 55°, and indicated with the respective angles γ₃′ and γ₃″. Together, they may define a total third circle section γ₃, here about 110°.

FIG. 2e VI schematically depicts a schematical, or more theoretical, illustration (like embodiment I of FIG. 2e does), of airflow channels 731,732, which is again depicted as tubes.

In embodiments, the substantially (radial) first airflow channel part 731a of the first airflow channel 731 may have a first channel part axis 740a of the first airflow channel 731 which may have a first angle θ1 with a plane SP perpendicular to the device axis A1. Further, the substantially (tangential) second part 731b of the first airflow channel 731 may have a second channel part axis 739a which may have a third angle θ3 with the first channel part axis 740a of the first airflow channel part 731a of the first airflow channel 731.

In embodiments, the substantially (radial) first airflow channel part 732a of the second airflow channel 732 may have a first channel part axis 740b of the second airflow channel 732 which may have a second angle θ2 with a plane SP perpendicular to the device axis A1. Further, the substantially (tangential) second part 732b of the second airflow channel 732 may have a second channel part axis 739b which may have a fourth angle θ4 with the first channel part axis 740b of the first airflow channel part 732b of the second airflow channel 732.

The first angle θ1 and the second angle θ2 may each individually be selected from the range of about 0-90°, like e.g. 0-85°, though other values may also be possible, like 0-180°. Especially, θ1+θ2>0°, such as θ1+θ2≥5°, such as θ1+θ2≥15°, like in embodiments 5°≤θ1+θ2≤2*85°, such as 10°≤θ1+θ2≤2*75°.

Further, in embodiments, 30°≤θ3≤175°, such as 45°≤θ3≤135°, like about 60°≤θ3≤135°, such as even more especially about 75°≤θ3≤135°. However, other angles are not excluded.

Yet further, in embodiments, 30°≤θ4≤175°, such as 45°≤θ4≤135°, like about 60°≤θ4≤135°, such as even more especially about 75°≤θ4≤135°. However, other angles are not excluded.

The first channel parts of the airflow channels 731,732 may have an inlet which receive airflow(s) from the airflow device. The inlets are indicated with references 737a and 737b, respectively. These inlets may be offset from the intersection of the device axis A1 and the plane perpendicular to SP. The first parts of the channels are indicated with references 731a. 732a, respectively, and the second parts of the channel are indicated with references 731b, 732b, respectively. Especially, the respective electrodes 510,520 are configured in the second parts 731b,732b, or directly upstream thereof. Note that in application embodiments the channel may have other shapes than schematically depicted, and may e.g. include curved bends, etc. Further, the channel may comprise fins or may be defined by fins (see also above). With such type of channels, a kind of (internal) swirl of the airflow is provided, leading to airflows that may leave in different directions from the device, with directions that may not be in the same plane, or when in other embodiments being in the same plane, the directions may not intercept the device axis A1. Especially, in embodiments airflows that may leave in different directions from the device, with directions that may not be in the same plane, as this may (even more) reduce annihilation of the charged particles when the first and second airflows are provided at the same time.

Referring to embodiment VI of FIG. 2e, schematically an embodiments is shown wherein at least part of the first airflow channel 731, even more especially at least a part 731b of the first airflow channel 731 downstream of the first electrode 510, and especially essentially the entire part downstream of the first electrode 510, thus including the first part 721 of the second openings 720, may be defined by channel walls, which when (virtually) elongated along a channel axis of such channel part downstream of the first electrode may not intersect with the device axis A1.

Likewise, in embodiments at least part of the second airflow channel 732, even more especially at least a part 732b of the second airflow channel 732 downstream of the second electrode 520, and especially essentially the entire part downstream of the second electrode 520, thus including the second part 722 of the second openings 720, may be defined by channel walls, which when elongated along a channel axis of such channel part downstream of the second electrode may not intersect with the device axis A1.

Hence, these channel axes 739a, 739b may thus (also) not be in the same plane, and may not coincide with the device axis A1.

Embodiment VI of FIG. 2e may schematically/theoretically depict how embodiments of the flows of embodiment I of FIG. 2c may be obtained.

FIG. 2f schematically depicts a further embodiment of the device 1200. Referring to e.g. FIG. 1, FIG. 2b embodiments I and II, FIG. 2d, embodiment I, and FIG. 3, in embodiments the first part 721 and/or the second part 722 may at least partly enclose the optical element 770. Hence, when viewed from the front, a viewer may perceive the first part and/or the second part of the second openings. Especially, however, the viewer may not perceive the first openings. In other embodiments, however, when viewed from the front, a viewer may perceive only part of the first part and/or the second part of the second openings (and the viewer may not perceive the first openings). This may e.g. be the case when the second openings are partly configured in a part of the device that tapers in a direction from the first end to the second end. In yet other embodiments, however, when viewed from the front, a viewer may not perceive the first part and/or the second part of the second openings (and the viewer may not perceive the first openings). This may e.g. be the case when the second openings are configured in a part of the device that tapers in a direction from the first end to the second end. For instance, when the front element (see also above) may hide the second openings for the viewer (viewed from the front).

Referring to FIG. 2f, in embodiments at least part of (the first part 721 and/or the second part 722 of) the second openings 720 may be configured behind an edge of the front part, such as an edge of the front element. The edge of the front part 750 is indicated with reference 751, and the edge of the front element 760, is indicated with reference 761. Note that in the schematically depicted embodiment, the edge 751 of the front part 750 may coincide with the edge 761 of the front element 760. Here, the front part 750 may comprise or may be the front element 760. Hence, in embodiments the second openings 720 may be located at the side of the device, near to the front.

Referring to FIG. 2f (and other schematically depicted embodiments), in specific embodiments, the front part 750) may have a part, closer to the first end 1210 that tapers in a direction from the second end 1220 to the first end 1210, and an adjacent part that tapers in a direction from the first end 1210 to the second end 1220. These two parts are indicated with the dashed line in the first part 750, above which the part, closer to the first end that tapers in a direction from the second end to the first end is depicted, and below which adjacent part that tapers in a direction from the first end to the second end is depicted.

Referring to e.g. FIG. 2a, embodiments I, III, and IV, FIG. 2b embodiments IV, FIG. 2e, embodiments IV and V, FIG. 2f (embodiment I), and also FIG. 3 the first part 721 may in embodiments comprise 1-4 second openings 720, and the second part 722 may in embodiments comprises 1-4 second openings 720. In yet further specific embodiments, the front part may have an essentially circular cross-section. The circle may be divided in a first circle section comprising the first part of the one or more second openings and a second circle section comprising the second part of the one or more second openings. Especially, in embodiments there may be a third circle section comprising not second openings. In embodiments, the first circle section and the second circle section are separated by the third circle section. The third circle section may comprise one or more third circle section. In embodiments, the total third circle section may have an angle of at least 90°, even more especially at least 135°, such as in specific embodiments at least about 180°. Hence, assuming in embodiments a single first circle section, and oppositely configured thereof a second circle section, these two section may be separated by two third circle sections. The latter may have angles of especially at least 45°.

Embodiment II of FIG. 2f schematically depicts a top view. Note that the second openings in this embodiment are not visible. By way of example, possible directions 1010,1020 of the airflows 610,620 are depicted.

Referring to FIG. 2g, in embodiments one or more of the following may apply: (a) a first airflow channel 731 configured between the airflow device 600 and the first electrode 510 comprises a curved shape, and (b) a second airflow channel 732 configured 20) between the airflow device 600 and the second electrode 520 comprises a curved shape.

Referring to FIG. 2g, but also to FIGS. 1 and 2d embodiment I, the device 1200 may comprise a support element 850, configured to support the first electrode 510 and the second electrode 520. Especially, the support element 850) may at least partly define one or more of the first airflow channel 731 and the second airflow channel 732.

The dashed rectangle indicates the air flow device 600. Relative to the plane of drawing, the air flow device 600 is over the plane of drawing.

Reference 860 refers to flow influencing elements, such as curved channel elements 861 or partitions 862,863, such as vanes 865. Hence, the airflow channels 731,732 may at least partly be defined by curved channel elements or with other (flow influencing) elements. The dashed areas at the left and the right may indicate (a wall of) an enclosure arrangement, such as a housing. The enclosure arrangement and the support element 850 may define part of the airflow channels 731,732. Hence, the support element 850) may be configured (at least partly) downstream of the airflow device 600 (here not depicted but the dashed square in the middle may indicate the position of the airflow device 600). The support element 850 may comprise an opening at least partly defining the respective flow channel. Alternatively or additionally, the support element together with an enclosure arrangement may define an opening at least partly defining the respective flow channel: see FIG. 2g, references 741a, 741b.

FIG. 2h schematically depict in embodiment(s) I a non-limiting number of shapes of fins 725. From left to right, the left one (a) may introduce more area with which the airflow may come into contact. Hence, this may lead to turbulence and premature annihilation of the charge of charged particles. The next fin 725 (b) may introduce less area with which the airflow may come into contact and may thus lead to less premature annihilation of charges. This fin 725 depicted at (b) may have a kind of wing shape. Hence, in embodiments one or more fins 725 may have a wing shape (see also embodiment d in FIG. 2h, embodiments I). The fin 725 depicted at (c) may further introduce a swirl effect and may also introduce less area with which the airflow may come into contact and may thus lead to less premature annihilation of charges. The fin schematically depicted at (d) may have a wing shape, with in embodiments a substantial round front part and a tapering end part, which wing may in embodiments have a total length in the range of about 2-9 times the diameter of the substantial round part.

Embodiments II-IV of FIG. 2h schematically depict some cross-sections of embodiments of openings 720. Hereby, the possible curved aspect of the opening along the device axis A1 (thus perpendicular to the plane of drawing) is ignored. Hence, the cross-sections may be cross-sections of embodiments of the openings 720 as if they were deformed to be straight/linear.

Embodiment II in FIG. 2h schematically depicts an embodiment of an opening 720 defined by fins 725. Here, by way of example curved fins 725 are applied, leading to an angle β1 with the device axis A1 which may vary over the height of the opening 720. Embodiment II of FIG. 2h may e.g. represent a second opening 720 of FIG. 2b, embodiment III, and may lead to flow directions as schematically depicted in FIG. 2c, embodiments II-III or FIG. 2e, embodiment IV.

Embodiment III in FIG. 2h schematically depicts an opening 720 defined by fins 725 which are configured parallel to the device axis A1. This may lead to flow directions essentially parallel to the device axis A1.

Embodiment IV in FIG. 2h schematically depicts an opening 720 defined by end edges 723. Here, by way of example curved end edges 723 are applied, leading to an angle β1 with the device axis A1 which may vary over the height of the opening 720. Embodiment II of FIG. 2h may e.g. represent a second opening 720 of FIG. 2a, embodiments I or III, or embodiment V of FIG. 2e, and may lead to flow directions as schematically depicted in FIG. 2c, embodiments II-III or FIG. 2e, embodiment IV.

FIG. 2i schematically depicts a non-limiting number of front views (of the first part 750). Embodiment I of FIG. 2i schematically depicts a front view of a front part 750 with two second openings 720, belonging to the first part 721 and second part 722, respectively.

Embodiment II of FIG. 2i schematically depicts a front view of a front part 750 with four second openings 720, two belonging to the first part 721 and two belonging to the second part 722, respectively. Here, the second openings are configured at different distances from the device axis A1.

Embodiment III of FIG. 2i schematically depicts a front view of a front part 750 with four second openings 720, two belonging to the first part 721 and two belonging to the second part 722, respectively. The first parts 721 and second parts 722 may be configured in a ++−− configuration. Other configurations, however, may also be possible.

Embodiment IV of FIG. 2i schematically depicts a front view of a front part 750 with two second openings 720, belonging to the first part 721 and second part 722, respectively. Here, the openings second 720 are configured radially: in embodiments I-III of FIG. 2i, the second openings 720 are approximately tangentially shaped.

Embodiment V of FIG. 2i schematically depicts a front view of a front part 750, wherein the first part 721 and the second part 722 comprises a plurality of second openings 720 separated by fins 725.

Embodiment VI of FIG. 2i schematically depicts a front view of a front part 750, wherein the first part 721 and the second part 722 are not visible, but (just) behind an edge (see also FIG. 2f, embodiment II).

Referring to FIG. 2j (see also FIG. 2e VI), an embodiment is show how the airflows may be generated. Here, also an embodiment of the internal swirl is schematically depicted.

FIG. 3 schematically depicts amongst others exploded views of an embodiment of the device, especially in embodiment I. Embodiment II shows a top view of a cross-section. Embodiment II is surrounded by four side views. Embodiments III (and IV) shows views of the device, with some parts made transparent to see other parts more within the device. Embodiments IV shows another cross-sectional view.

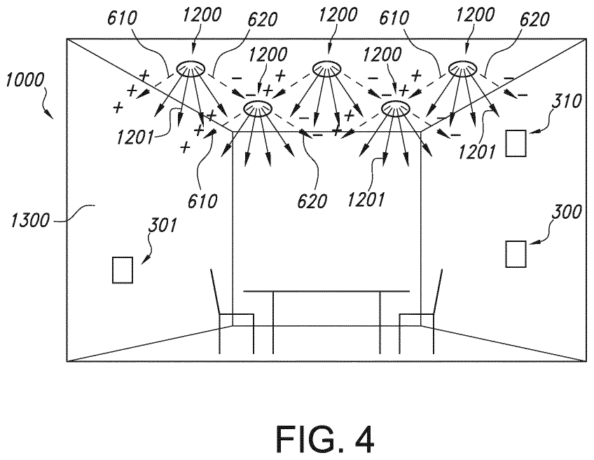

FIG. 4 schematically depicts an embodiment of a light generating system 1000 comprising an embodiment of the device 1200. The light generating system 1000 may further comprise a control system 300. Reference 301 indicates a user interface which may be functionally coupled with the control system 300 comprised by or functionally coupled to the light generating system 1000. Reference 310 schematically depicts a sensor, which may be functionally coupled to the control system 300. The sensor 310 may be comprised by the light generating system 1000. Further, one or more of the devices 1200 may comprise a sensor (not depicted). Especially, the control system 300 may be configured to control the device 1200 in dependence of one or more of an input signal of a user interface, a sensor signal, and a timer. Especially, the control system 300 may be configured to control a plurality of the devices 1200 (in dependence of one or more of an input signal of a user interface, a sensor signal, and a timer). In specific embodiments, one or more of the devices may comprise the control system 300.

FIG. 4 schematically depicts an embodiment of the system 1000 comprising a grid of air devices 1200. In specific embodiments, the devices 1200 may be downlighters or spotlights. Here a space 1300 is shown, such as an office or living room, etc.

Hence, amongst others the invention provides a method for treating at least part of a space 1300, wherein the method comprises providing the first airflow 610 and/or a second airflow 620 in the space 1300. Alternatively or additionally, the method may comprise providing device light 1201 in the space 1300.

Referring to e.g. FIGS. 1, 3 and 4, the invention may thus also provide a method for one or more of (i) treating a gas and (ii) providing light, wherein the method comprises operating the device 1200 or the light generating system 1000.

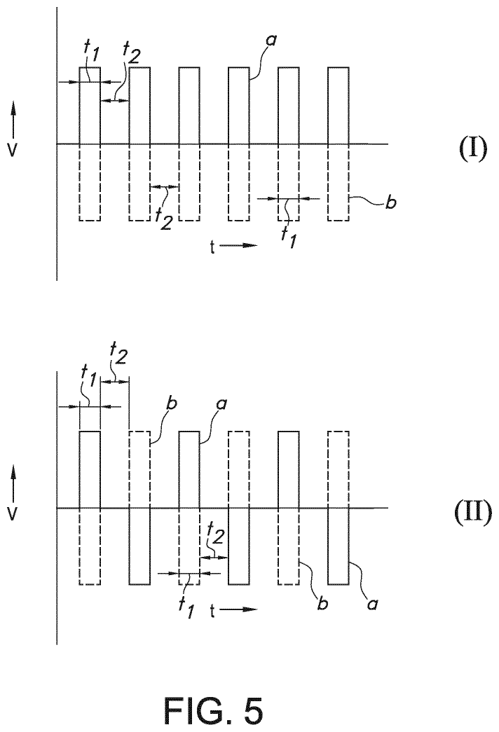

FIG. 5 schematically depicts non-limiting embodiments of how the electrodes may be operated. In FIG. 5, embodiments I, curve a may e.g. refer to the voltage-time scheme applied to the first electrode, and curve b may e.g. refer to the voltage-time scheme applied to the second electrode. The periods the potential differences are applied to the electrodes are the same, and always of the same sign.

In embodiments II of FIG. 5, the first electrode and the second electrode are subjected to alternating voltages, but during the same on-time periods t1. The period of time between the pulses is indicated with the off-time period t2.

With such pulsating voltages, at least two different airflows may be provided, one temporarily entraining positive particles and one temporarily entraining negative particles, which in these schematically depicted embodiments provided at the same time (i.e. fully overlapping pulse periods).

Note that in other embodiments the electrodes may also be operated continuously (i.e. not a pulsed voltage).

The term “plurality” refers to two or more.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage. In yet a further aspect, the invention (thus) provides a software product, which, when running on a computer is capable of bringing about (one or more embodiments of) the method as described herein.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A device comprising (i) a light generating device, (ii) an air ionizer device, and (iii) an airflow device, wherein:
the device comprises one or more first openings and a front part comprising one or more second openings;
the air ionizer device is configured to generate in an operational mode positively charged particles at a first electrode and negatively charged particles at a second electrode;
the airflow device is configured to generate in the operational mode a first airflow entraining the positively charged particles and a second airflow entraining the negatively charged particles; wherein the one or more first openings are configured upstream of the airflow device and the electrodes; wherein a first part of the one or more second openings is configured downstream of the airflow device and the first electrode, and a second part of the one or more second openings is configured downstream of the airflow device and the second electrode;
the device comprises a light emitting area, from which during operation of the light generating device device light escapes from the device, wherein the front part comprises the light emitting area;
the device comprises one or more solid state light sources and a front element, wherein the front element comprises an optical element, wherein the optical element is configured downstream of the one or more solid state light sources, wherein the first part and the second part at least partly enclose the optical element;
wherein the front element comprises a central part and a peripheral part, wherein the central part comprises the optical element, wherein the peripheral part comprises the first part and the second part.

2. The device according to claim 1, wherein the optical element comprises a diffuser.

3. The device according to claim 2, wherein reflectivity of the diffuser is in the range from 20 to 60% from the light of the one or more (solid state) light sources.

4. The device according to claim 1, wherein the optical element is at least partly surrounded by the first part and the second part; wherein the first part comprises 1-4 second openings, and wherein the second part comprises 1-4 second openings.

5. The device according to claim 1, wherein the front element is a monolithic element.

6. The device according to claim 1, comprising a device axis, wherein the first airflow has a first direction having a first angle relative to the device axis, and wherein the second airflow has a second direction having a second angle relative to the device axis.

7. The device according to claim 6, wherein the first angle and the second angle are independently selected from the range of 0-90°, wherein the first direction and the second direction are not configured in a single straight plane and their mutual angle is selected from the range of between 5-180°.

8. The device according to claim 6, wherein the first direction and the second direction have a mutual angle selected from the range of 5°-150°.

9. The device according to claim 6, wherein one or more of the following applies: (a) the first part comprises at least one second opening at least partly defined by fins, which are not in a same plane as the device axis and which are non-perpendicular to a plane perpendicular to the device axis, and (b) the second part comprises at least one second opening at least partly defined by fins, which are not in a same plane as the device axis and which are non-perpendicular to a plane perpendicular to the device axis.

10. The device according to claim 6, wherein one or more of the following applies: (a) a first airflow channel configured between the airflow device and the first electrode comprises curved shape, and (b) a second airflow channel configured between the airflow device and the second electrode comprises a curved shape.

11. The device according to claim 10, comprising a support element, configured to support the first electrode and the second electrode, wherein the support element at least partly defines one or more of the first airflow channel and the second airflow channel.

12. The device according to claim 11, wherein the device comprises an enclosure arrangement, wherein the enclosure arrangement at least partly encloses the light generating device, the air ionizer device, and the airflow device; wherein the device comprises a first end and a second end; wherein the light emitting area is configured closer to the first end than to the second end; wherein the enclosure arrangement comprise the or more first openings configured between the first end and the second end; wherein the device comprises electrical connectors and electronics, wherein the electronics are at least partly enclosed by the enclosure arrangement, wherein the electronics are functionally coupled to the light generating device, the air ionizer device, the airflow device, and the electrical connectors, and wherein the electrical connectors are configured closer to the second end than the first end; and wherein one or more of the enclosure arrangement and the support element comprises one or more flow influencing elements, wherein the one or more flow influencing elements are at least partly configured in at least one of the first airflow channel and the second airflow channel.

13. The device according to claim 1, wherein the device is a retrofit lamp; and wherein the device in an operational mode is configured to generate white device light.

14. A light generating system comprising the device according to claim 1, wherein the light generating system comprises a control system configured to control the device in dependence of one or more of an input signal of a user interface, a sensor signal, and a timer.

15. A method for one or more of (i) treating a gas and (ii) providing light, wherein the method comprises operating the device according to claim 1 or the light generating system.
